(19) United States
(12) Patent Application Publication
(10) Pub. No.: US 2017/0081647 A1
(43) Pub. Date: Mar. 23, 2017

(54) GENETIC DETECTION PLATFORM

(71) Applicant: Pharmozyme, Inc., Sunnyvale, CA (US)

(72) Inventor: Nader Nouri Zad, Sunnyvale, CA (US)

(21) Appl. No.: 15/311,493

(22) PCT Filed: May 15, 2015
(86) PCT No.: PCT/US15/31196

§ 371 Date: Nov. 15, 2016

Related U.S. Application Data
(60) Provisional application No. 61/994,829, filed on May 16, 2014.
(60) Provisional application No. 62/044,872, filed on Sep. 2, 2014.

(51) Int. Cl. C12N 9/12; C12N 15/10; C12P 19/34; C12Q 1/68

(52) U.S. Cl. CPC C12N 9/1252; C12Q 1/686; C12Y 207/07007; C12N 15/1006; C12P 19/34

(57) ABSTRACT

Disclosed herein are methods, compositions, assays, and kits for performing polynucleotide amplification utilizing a pure polynucleotide polymerase. Also disclosed herein are methods, compositions, assays, and kits for removal of nucleic acid contaminant from the polynucleotide polymerase.

### CROSS-REFERENCE

This application claims the benefit of U.S. Provisional Application Nos. 61/994,829 filed May 16, 2014 and 62/044,872 filed Sep. 2, 2014, which are incorporated herein by reference in their entirety.

### REFERENCE TO A SEQUENCE LISTING SUBMITTED VIA EFS-WEB

The content of the ASCII text file of the sequence listing named “46681-701-601-seqlist_ST25.txt” which is 17 kb in size was created on May 14, 2015, and electronically submitted via EFS-Web herewith the application is incorporated herein by reference in its entirety.

### BACKGROUND OF THE INVENTION

Genetic amplification and genetic sequencing have gained considerable interest in recent years. Various methodologies for genetic amplification have been developed to facilitate such amplification. Generally, the amplification of polynucleotides (such as DNA) requires a polynucleotide strand to be amplified (target polynucleotide), short polynucleotide fragments containing sequences complementary to the target (i.e. a primer), nucleotides and an enzyme that polymerizes (i.e. covalently links) the nucleotides in a manner complementary to the target polynucleotide.

One popular amplification reaction is the polynucleotide chain reaction (PCR), which employs a heat-stable DNA polymerase. An example of such a polymerase is Taq-polymerase, which was originally isolated from the bacterium Thermus aquaticus. PCR relies on thermal cycling, consisting of cycles of repeated heating and cooling of the reaction for DNA melting and enzymatic replication of the DNA. The thermal cycling is required for (a) denaturation: separation of double stranded a polynucleotide into single stranded polynucleotides which serve as a template for the association of the nucleotide that would form the complementary strand, which typically occurs at 94-98° C.; (b) annealing: lowering the temperature to allow hydrogen bonding of the primer to the separated polynucleotide strand (i.e. ssDNA), which typically occurs at 50-65° C.; and (c) elongation: allowing optimal condition for enzymatic polymerization, thus forming the complementary strand to the template polynucleotide, which typically occurs at 70-80° C. (depending on the particular polymerase used).

Currently, genetic detection has limited sensitivity due to contaminants that are present in amplification reagents (such as polynucleotide contaminants from the host genomic material and plasmid).

### SUMMARY OF THE INVENTION

Methods, compositions, reagents, enzymes, kits, programs, business methods, and reports are provided herein for polynucleotide amplification enzymes, sample preparation for their expression, use in amplification, sequencing, nucleic acid contaminant removal, amplification enzyme activity characterization, or any combination thereof.

In some aspects, the invention discloses a polymerase construct comprising at least one moiety that is capable of binding albumin. Sometimes, the polymerase is Taq polymerase. The Taq polymerase may be a native or modified Taq polymerase described herein. The Taq polymerase may have the sequence of SEQ ID NO: 1.

In some aspects, the invention discloses a composition comprising a polymerase comprising at least one albumin binding moiety.

In some aspects, the invention discloses a reaction mixture which comprises (a) a polymerase comprising an albumin binding moiety; and (b) an albumin.

In some aspects, the invention also discloses a method for amplifying a target DNA which comprises (a) incubating the target DNA with a polymerase having the polymerase construct described herein, an albumin, a set of primers, and nucleoside polyphosphates selected from the group consisting of adenine, thymine, guanine, cytosine, and uridine; so as to form a reaction mixture; and (b) subjecting the reaction mixture to an amplification method, whereby the set of primers is extended by the polymerase to amplify the target DNA sequence.

In some aspects, the invention discloses an albumin affinity purification method for enzyme production which comprises (a) forming a protein construct comprising a target polymerase bound to an albumin binding moiety; (b) contacting the protein construct with albumin to form an albumin molecular complex; (c) separating the protein construct; and (d) retrieving the protein construct from the albumin molecular complex, wherein the protein construct retains activity of the target enzyme.

In some aspects, the invention further discloses a method of removing a nucleic acid contaminant from a biological sample, which comprises (a) contacting a biological sample with protamine-coated beads; and (b) harvesting the biological sample from protamine-coated beads through a separation method to remove the nucleic acid contaminant from the biological sample.

In some aspects, the invention discloses an assay kit for determining the activity of a polymerase comprising an oligonucleotide selected from SEQ ID NOs: 8-10.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an overview of a method, a system, and an apparatus disclosed herein.

FIG. 2 shows a purification method disclosed herein. Titles on columns designate the target entity (i.e. purification tag) that the column has affinity for.

FIG. 3 depicts constructs of the modified Taq-polymerase disclosed herein.

FIG. 4 depicts additional constructs of the modified Taq-polymerase disclosed herein.

FIG. 5 illustrates various albumin binding sites in Streptococcal Protein G.

FIG. 6 illustrates a Taq polymerase described herein binding to human serum albumin (HSA).

FIG. 7 illustrates a diagram of the computer system disclosed herein.

FIG. 8A-FIG. 8F show gel electrophoresis of the Taq polymerase-ABS construct following purification from either E. coli or yeast cells.

FIG. 9A and FIG. 9B illustrate a luminescence assay that indicates the Taq polymerase activity. As shown in FIG. 9A and FIG. 9B, a time course is initially indicated with a lag phase, normally less than 2 minutes, followed by a linear increase in luminescence. The rate of luminescence increase (slope) defines the Taq polymerase activity.

FIG. 10 depicts various Z domain designs described herein.

### DETAILED DESCRIPTION OF THE INVENTION

Methods, compositions, reagents, enzymes, kits, programs, business methods, and reports are provided herein for polynucleotide amplification enzymes, sample preparation for their expression, use in amplification, sequencing, nucleic acid contaminant removal, amplification enzyme activity characterization, or any combination thereof. The methods, compositions and reagents find use in a number of applications, including, for example in polynucleotide sample preparation for their expression, amplification, sequencing, or any combination thereof. In addition, devices, systems, kits, programs, business methods, reports and computer software thereof may find use in practicing the subject methods, and may use the compositions and reagents provided. These and other objects, advantages, and features of the invention will become apparent to those persons skilled in the art upon reading the details of the methods, compositions, reagents, devices, systems, kits, programs, business methods, reports and computer software as more fully described below.

Before the present methods, compositions, reagents, enzymes, devices, systems, kits, programs, business methods, reports or computer software are described, it is to be understood that this invention is not limited to the particular methods, compositions, reagents, devices, systems, kits, programs, business methods, reports or computer software described, as such may, of course, vary. It is also to be understood that the terminology used herein is for the purpose of describing particular embodiments only, and is not intended to be limiting, since the scope of the present invention will be limited only by the appended claims.

Where values are provided, it is understood that each value is accurate to the tenth of the unit (i.e. +/−0.1 unit) unless the context clearly dictates otherwise. Each smaller range between any stated value or intervening value in a stated range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a cell” includes a plurality of such cells, reference to “the polynucleotide” includes reference to one or more polynucleotides and equivalents thereof; and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### General Description

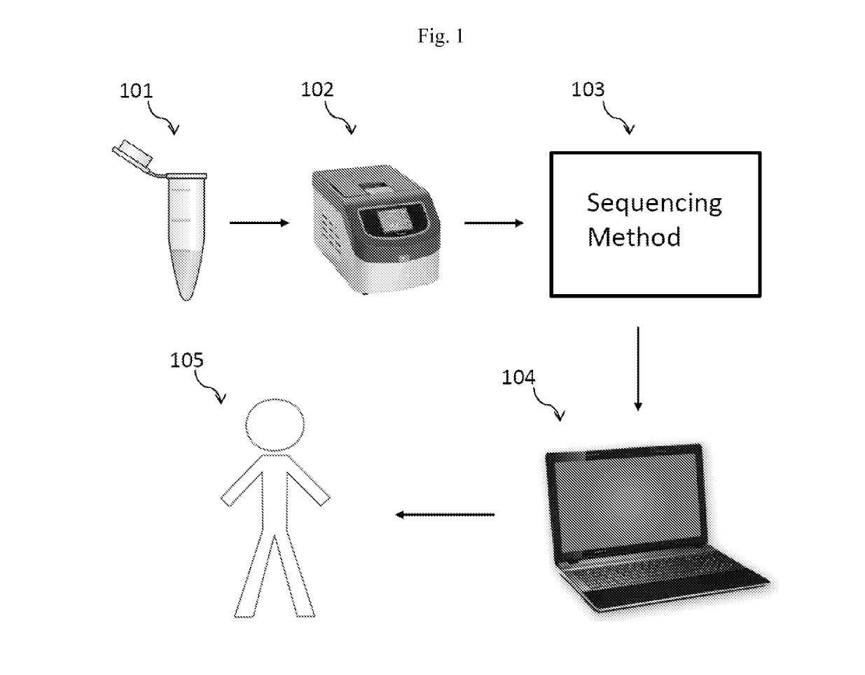

Disclosed herein are methods, compositions, apparatus, and kits for sequencing nucleic acid on an efficient scale and with low contaminations and false positives. The specification facilitates amplification and/or sequencing of a target sample containing target polynucleotides (e.g. DNA) from a subject as illustrated in FIG. 1. The sample may contain any sample that contains the target polynucleotide. The sample may be a bodily sample such as any bodily fluid, body part or tissue. For example, the sample may comprise blood, hair, skin, amniotic fluid, cells (e.g. cheek cells). The subject may be a human or an animal. The animal can be a mammal. The animal can be a pet, a wild animal, a farm animal or a laboratory animal. In some examples, the sample is inserted into a polynucleotide amplifier. The sample can subsequently undergo polynucleotide sequencing in a polynucleotide sequencer with the use of a polymerase described herein. Sometimes, sequencing reaction mixture further comprises an albumin which inactivates the polymerase at a non-polymerase extension temperature. At a polymerase extension temperature, the albumin may become inactivated, thereby restoring the activity of the polymerase. The data from the polynucleotide sequencer can be transmitted. The data from the polynucleotide sequencer can be further analyzed. The raw or analyzed data can be delivered, transmitted, or reported to the requesting party. The requesting party may be the subject, a laboratory, a governmental entity, a hospital, a law enforcement facility, a physician, a health related facility, or any requesting party. The sample may be amplified and/or sequenced in exchange for a fee. The raw or analyzed data can be delivered, transmitted, or reported in exchange for a fee.

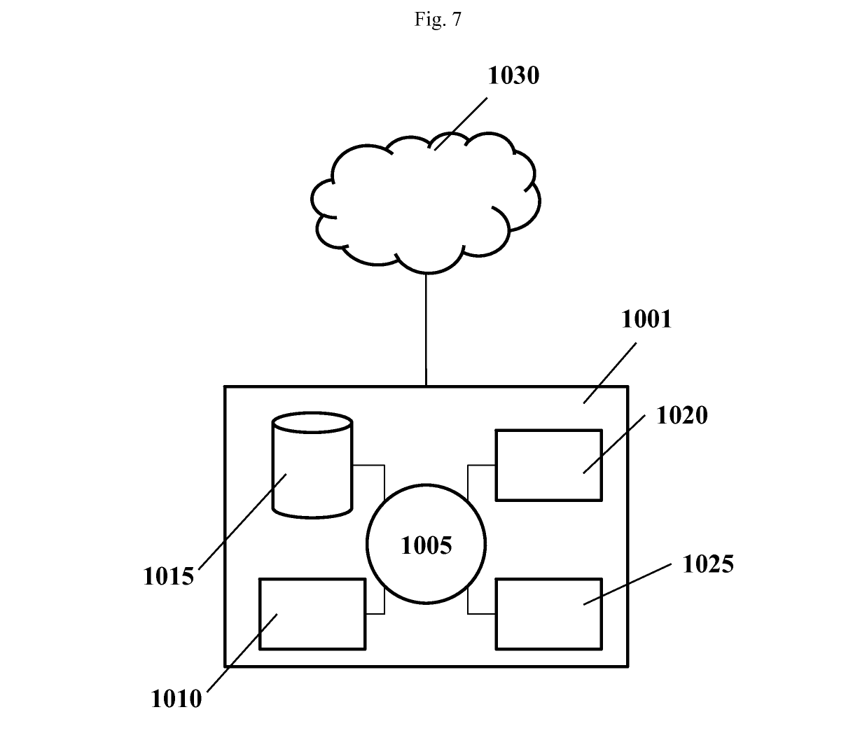

In some examples, 101 illustrates a sample input for the DNA sequencing device 102, in which the sample input comprises a polymerase described herein and/or a polymerase treated by a nucleic acid contaminant removal step described herein. 101 further contains reagents and components that facilitate the sequencing reaction, such as for example, albumin. 102 carries out a sequencing method described herein 103. In some cases, 102 further comprises a luminometer capable of measuring the production of light during the reaction. Upon completion of 103, the results can be transmitted to a computer 104. In some cases, 104 is as described in FIG. 7. 105 can be operatively coupled to a computer network (e.g. Internet, an internet, extranet, telecommunication network, or a data network). 104 can contain an electronic storage system. In some cases, the electronic storage system include non-transitory storage modules such as any or all of the tangible memory of the computers, processors or the like, or associated modules thereof, such as various semiconductor memories, tape drives, disk drives and the like; or an external storage devices, such as for example, hard disks, external hard drives, CDs, DVDs, flash drives, or the like. 104 can analyze the data, and can transmit the data to a user 105. The transmission of the data can be via the computer network (e.g. Internet, an internet, extranet, telecommunication network, or a data network). The transmission of the data can be via the electronic storage system (e.g. non-transitory storage modules or externally storage devices). The data can be transmitted visually, such as for example, shown on a screen that is part of 104 or as an externally connected screen, or by sound. The user 105 can be an operator or an end user. In some cases, the end user is a lab technician, a physician, a patient, a researcher, or a customer.

The sequencing method benefits from a polymerase that is substantially pure. The polymerase may be at least about 95%, 96%, 97%, 98%, 99%, 99.5%, or 99.99% pure. The polymerase may be at most about 95%, 96%, 97%, 98%, 99%, 99.5%, or 99.99% pure. The polymerase may be without nucleic acid contaminant or substantially without nucleic acid contaminant. The polymerase may be free of nucleic acid contaminant, or substantially free of nucleic acid contaminant.

The sequencing method benefits from a polymerase which can interact with a polymerase inhibitor at a temperature that is lower than the polymerase extension temperature but will be released from the interaction of the polymerase inhibitor at or above the polymerase extension temperature. The polymerase inhibitor can be an albumin. In some aspects, the inhibition of a polymerase (e.g., Bio-HIS-ABS-Taq or Bio-HIS-Z domain-Taq described herein) by the addition of albumin (e.g., human serum albumin) can be referred to as a Hot Start process. The combination of a polymerase (e.g., Bio-HIS-ABS-Taq or Bio-HIS-Z domain-Taq described herein) and albumin (e.g., human serum albumin) can be referred to as a Hot Start mixture. The polymerase (e.g., Bio-HIS-ABS-Taq or Bio-HIS-Z domain-Taq described herein) can be referred to as a Hot Start polymerase.

The sequencing method may be facilitated by a modified polymerase. The modified polymerase may be a DNA polymerase or an RNA polymerase. The modified polymerase may be a DNA polymerase. Sometimes, the modified polymerase is modified Taq polymerase. The modification may be chemical modification. The modification may be an enzymatic construct that incorporates a protein able to bind albumin and Taq polymerase. Such albumin may be bovine serum albumin or human serum albumin. The sequencing method may be any sequencing method employed in the art. Additionally, the sequencing method may incorporate unique enzyme constructs or modified enzymes as explained herein.

One or more of the abovementioned polymerases can be modified to incorporate a HIS construct comprising from about 4 to about 14 or from about 6 to about 12 histidine moieties. One or more of the abovementioned polymerases can be modified to incorporate an albumin binding domain (i.e. ABS). One or more of the abovementioned polymerases can be modified to incorporate a Biotin tag (Bio) or a biotin binding domain tag. One or more of the abovementioned polymerases can be modified to incorporate both HIS (e.g., HIS(6)) moiety and an albumin binding domain. One or more of the abovementioned polymerases can be modified to incorporate HIS (e.g., HIS(6)) moiety, an albumin binding domain, and a Biotin tag (e.g., a biotin binding domain tag). One or more of the abovementioned polymerases can be modified to incorporate a Z domain or Z domain moiety (see infra). One or more of the abovementioned polymerases can be modified to incorporate a modified albumin binding domain. One or more of the abovementioned polymerases can be modified to incorporate both Z domain and an albumin binding domain. One or more of the abovementioned polymerases can be modified to incorporate ABP-Z. One or more of the abovementioned polymerases can be modified to incorporate a Z domain (or Z domain moiety) in place of an albumin binding domain. Taq polymerase may be thus modified.

ABP-Z is a modified ABP that is able to bind protein A. The ABP-Z protein is an albumin binding protein that further contains a second binding site which recognizes a Z domain (or Z domain moiety) from SpA. In the Z domain (or Z domain moiety), at most 1, 2, 5, 8, 10, 11, 12, or 15 amino acid residues on ABP can be genetically modified to generate the second binding site. Sometimes, about 11 amino acid residues are genetically modified in ABP. The amino acid residues corresponding to residue position 22, 25, 26, 29, 30, 33, 34, 54, 57, 58 and 62 can be genetically modified to generate the second binding site that recognizes the Z domain.

### Taq Polymerase

In some aspects, the invention includes an amplification enzyme for use in a method disclosed herein. The amplification enzyme can be a polymerase. The polymerase can be a Taq polymerase. The Taq polymerase can be a native Taq polymerase, or a modified Taq polymerase. As used herein, Taq polymerase refers to any Taq polymerase, (native Taq polymerase or modified Taq polymerase). The Taq polymerase can be a Taq polymerase that is free from contamination (e.g. polynucleotide contamination), which is fabricated by custom engineering of Taq polymerase and producing in a cell. The custom engineering may include custom engineering of the genetic sequence encoding the custom engineered Taq polymerase protein, custom engineering the Taq polymerase protein, or a combination thereof. The cell (i.e. host cell) may include any suitable cell such as naturally derived cell or a genetically modified cell. The host cell may be a eukaryotic cell or a prokaryotic cell. An eukaryotic cell may include fungi, animal cell or plant cell. In some instances, the eukaryotic cell includes yeast. Sometimes, the yeast is a yeast capable of digesting polysaccharides into carbon dioxide and ethanol. Sometimes the yeast is baker's yeast or brewer's yeast or wine yeast (e.g. Zygosaccharomyces or Brettanomyces). Brewer's yeast may include Saccharomyces cerevisiae, Saccharomyces pastorianus (formerly known as S. carlsbergensis), Brettanomyces bruxellensis, Brettanomyces anomalus, Brettanomyces custersianus, Brettanomyces naardenensis, Brettanomyces nanus, Dekkera bruxellensis or Dekkera anomala. Sometimes, the yeast is Pichia pastoris. Other yeast species are delineated below. The prokaryotic cell can be bacterial cell. A bacterial cell may be a gram-positive bacterium or a gram-negative bacterium. Sometimes the gram-negative bacteria is anaerobic, rod-shaped, or both. In some instances, the gram-negative bacterium is Escherichia coli (i.e. E. coli). Animal cells may include a cell from a vertebrate or from an invertebrate. An animal cell may include a cell from a marine invertebrate, fish, insects, amphibian, reptile, or mammal.

The gram-positive bacteria may be Actinobacteria, Firmicutes or Tenericutes. The gram-negative bacteria may be Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria may be Acidobacteria, Chloroflexi, Chrysiogenetes, Cyanobacteria, Deferribacteres, Dictyoglomi, Thermodesulfobacteria or Thermotogae. A bacterial cell bacterial may be Escherichia coli, Clostridium botulinum or Coli bacilli.

Fungi include ascomycetes such as yeast, mold, filamentous fungi, basidiomycetes, or zygomycetes. Yeast may include Ascomycota or Basidiomycota. Ascomycota may include Saccharomycotina (true yeasts, e.g. Saccharomyces cerevisiae (baker's yeast)) or Taphrinomycotina (e.g. Schizosaccharomycetes (fission yeasts)). Basidiomycota may include Agaricomycotina (e.g. Tremellomycetes) or Pucciniomycotina (e.g. Microbotryomycetes).

Yeast or filamentous fungi may include Saccharomyces, chizosaccharomyces, Candida, Pichia, Hansenula, Kluyveromyces, Zygosaccharomyces, Yarrowia, Trichosporon, Rhodosporidi, Aspergillus, Fusarium, or Trichoderma. The Yeast or filamentous fungi may include Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium tore-Aspergillus niger, Aspergillus nidulans, Aspergillus awamori, Aspergillus oryzae, or Trichoderma reesei. Yeast may also include Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii or Saccharomyces boulardii. Sometimes, the yeast is Pichia pastoris. Sometimes, the yeast is saccharomyces cerevisiae. Sometimes, the yeast is saccharomyces cerevisiae S288c (NP_013551.1).

Cells may be of a mollusk, arthropod, annelid or sponge. The mammalian cell may be of a primate, ape, equine, bovine, porcine, canine, feline or rodent. The mammal may be a primate, ape, dog, cat, rabbit or ferret. The rodent may be a mouse, rat, hamster, gerbil, hamster, chinchilla, fancy rat, or guinea pig. The bird cell may be of a canary, parakeet or parrots. The reptile cell may be of a turtles, lizard or snake. The fish cell may of a tropical fish. The fish cell may be of a zebrafish (e.g. Danino rerio). In some instances the cell may be of a nematode (e.g. C. elegans). The amphibian cell may be of a frog. The arthropod cell may be of a tarantula or hermit crab.

Cells may be derived from knock-out or knock-in versions of the aforementioned species may also be used. Engineering may include the use of genetic vectors such as PIC-9. The vectors may comprise one or more polynucleotide that encodes for at least the following two proteins: DNA polymerase and albumin. DNA polymerase may include the polymerase from Thermus aquaticus (Taq polymerase), Terminal deoxynucleotidyl transferase (TdT) (also known as DNA nucleotidylexotransferase (DNTT) or terminal transferase), Reverse transcriptase (RT), or any other polynucleotide polymerase known in the art. Additional polymerases include, but are not limited to, Bst DNA polymerase, Bsu DNA polymerase, Crimson Taq DAN polymerase, Deep Vent_{R}™ DNA polymerase, Deep Vent_{R}™ (exo-) DNA polymerase, E. coli DNA polymerase I, Klenow fragment (3′-5′ exo-), DNA polymerase I (large Klenow fragment), LongAmp® Taq DNA polymerase, LongAmp® Hot Start Taq DNA polymerase, M-MuLV reverse transcriptase; One Taq® DNA polymerase, One Taq® Hot Start DNA polymerase, phi29 DNA polymerase, Phusion® Hot Start Flex DNA polymerase, Phusion® High-Fidelity DNA polymerase, Q5®+Q5® Hot Start DNA polymerase, Sulfolobus DNA polymerase IV, T4 DNA polymerase, MTP™ Taq DNA polymerase, Theminator™ DNA polymerase, Vent_{R}™ DNA polymerase, and Vent_{R}™ (exo-) DNA polymerase.

DNA polymerase may be of the genus Thermus, Bacillus, Thermococcus, Pyrococcus, Aeropyrum, Aquifex, Sulfolobus, Pyrolobus, or Methanopyrus. DNA polymerase may include the polymerase from the species Thermus aquatics, Thermus thermophilus, Bacillus stearothermophilus, Aquifex pyrophilus, Geothermobacterium ferrireducens, Thermotoga maritime, Thermotoga neopolitana, Thermotoga petrophila, Thermotoga naphthophila, Acidianus infernus, Aeropyrum pernix, Archaeoglobus fulgidus, Archaeoglobus profundus, Caldivirga maquilingensis, Desulfurococcus amylolyticus, Desulfurococcus mobilis, Desulfurococcus mucosus, Ferroglobus placidus, Geoglobus ahangari, Hyperthermus butylicus, Ignicoccus islandicus, Ignicoccus pacificus, Methanococcus jannaschii, Methanococcus fervens, Methanococcus igneus, Methanococcus infernus, Methanopyrus kandleri, Methanothermus fervidus, Methanothermus sociabilis, Palaeococcus ferrophilus, Pyrobaculum aerophilum, Pyrobaculum calidifontis, Pyrobaculum islandicum, Pyrobaculum oguniense, Pyrococcus furiosus, Pyrococcus abyssi, Pyrococcus horikoshii, Pyrococcus woesei, Pyrodictium abyssi, Pyrodictium brockii, Pyrodictium occultism, Pyrolobus fumarii, Staphylothermus marinus, Stetteria hydrogenophila, Sulfolobus solfataricus, Sulfolobus shibatae, Sulfolobus tokodaii, Sulfophobococcus zilligii, Sulfurisphaera ohwakuensis, Thermococcus kodakaraensis, Thermococcus celer, Thermococcus litoralis, Thermodiscus maritimus, Thermofilum pendens, Thermoproteus tenax, Thermoproteus neutrophilus, Thermosphaera aggregans, Vulcanisaeta distributa, or Vulcanisaeta souniana.

Albumin may include native or genetically modified albumin. Albumin may include serum albumin. Serum albumin protein may include mammalian serum albumin. Mammalian serum albumin may include human serum albumin or bovine serum albumin. Human serum albumin or bovine serum albumin maybe produced in bacteria (e.g., E. coli). Human serum albumin or bovine serum albumin maybe produced in yeast (e.g. Pichia pastoris).

Vectors may include any suitable vectors derived from either an eukaryotic or prokaryotic sources. Vectors may be from bacteria (e.g. E. coli), insects, yeast (e.g. Pichia pastoris), or mammalian source. Bacterial vectors may include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2. In some instances the vector is pET21 from E.coli. Insect vector may include pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2. Yeast vectors may include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector. In some examples, the vector is pPIC9 from Pichia pastoris. Mammalian vectors may include transient expression vectors or stable expression vectors. Mammalian transient expression vectors may include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some examples, the vector construct is engineered such that when the sequence carried by the vector is expressed into a protein, the protein corresponding to a genetic code of a protein able to bind albumin (e.g., serum albumin) and the protein corresponding to the polymerase (e.g., Taq polymerase) are covalently linked. The produced albumin binding protein-modified polymerase may result, among others, in amplifying a polynucleotide without using anti polymerase antibody, while obtaining a clean amplification product. The protein able to bind albumin may be derived from Streptococcal Protein G. The protein binding albumin can incorporate at least one of ABD1, ABD2 and ABD3 domains. The albumin may be serum albumin. The polymerase may be Taq-polymerase.

In some examples, the covalent linkage between the protein able to bind albumin and the protein polymerase (e.g. Taq-polymerase) will allow the polymerase to be modified with the protein able to bind albumin at the n-terminus of the Taq polymerase. In some instances, the covalent linkage can allow the protein polymerase to be modified with the albumin binding protein at the N-terminus, C-terminus or both N and C termini. Sometimes, the covalent linkage between the polymerase and the protein able to bind albumin is through a direct covalent linkage. Occasionally, the covalent linkage between the polymerase and the protein able to bind albumin is through a spacer molecule linkage. The spacer molecule may be at least 1, 2, 3, 4, 5, 6, 7, 8, 9, 10 or more covalently linked amino acids. The spacer molecule may be a chimeric peptide, an organic molecule, saccharide, a peptide, a polynucleotide or a nucleic acid monomer. The organic molecule may be aliphatic, conjugated or aromatic. The conjugated organic molecule may comprise at least 1, 2, 3, 4, 5, 6, 7, 8, 9, 10 or more conjugated bonds. The saccharide may be a mono, di, oligo or poly saccharide. The polymerase may be Taq-polymerase. The albumin may be serum albumin or human serum albumin. At times, the albumin is bovine serum albumin.

The invention describes method for the production of polymerase (e.g., Taq-polymerase) in host cells. These methods may comprise modified or engineered Taq-polymerase and anti Taq polymerase in any of the above-mentioned host cells. In some examples, the invention describes method for the production of Taq polymerase in host cells (e.g., yeast or bacteria). In some examples, the invention describes method for the production of Taq polymerase in yeast cells (e.g., Pichia pastoris). These methods may comprise modified or engineered Taq polymerase and anti Taq polymerase in host cells. In some instances, the Taq polymerase described herein is obtained from an extracellular portion of the host cells. In some instances, the Taq polymerase described herein is obtained from an intracellular portion of the host cells.

The invention may comprise methods for high throughput protein production in a small, large or medium scale. These methods may comprise production of multiple proteins simultaneously in any of the abovementioned host cells. In some examples, the methods may comprise production of multiple proteins simultaneously in yeast or bacteria. Sometimes, the yeast is Pichia pastoris. Sometimes, the yeast is Saccharomyces cerevisiae. Sometimes, the yeast is Saccharomyces cerevisiae S288c (NP_013551.1). At times, the bacterium is Escherichia coli.

The modified polymerase (e.g., Taq-polymerase) construct incorporating the protein able to bind albumin may have a higher processivity than a polymerases without a protein able to bind albumin. As used herein, processivity is the average number of nucleotides added by the polymerase prior to the polymerase's dissociation from the DNA. The Taq polymerase construct incorporating the protein able to bind albumin may have a processivity rate of at least 1×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, or higher compared to non-albumin modified polymerases.

The polymerase (e.g., Taq-polymerase) construct incorporating the protein able to bind albumin may have a faster extension rate than polymerases that does not comprise a protein able to bind albumin. As used herein, extension rate is the maximum number of nucleotides polymerized per second per molecule of polymerase (e.g. DNA polymerase). The polymerase (e.g. Taq-polymerase) construct incorporating a protein sequence able to bind albumin may have an extension rate of at least 10, 11, 12, 13, 14, 15, 16, 17, 18, 19, 20, 25, 30, 35, 40, 45, 50, 55, 60, 90, 120 sec/kilobase, or more.

The polymerase (e.g., Taq-polymerase) construct that incorporates a protein sequence able to bind albumin may have higher fidelity than non-albumin modified polymerases. As used herein, fidelity is the ability of the polymerase to faithfully replicate a DNA molecule. Fidelity may be described by the rate of error. The Taq polymerase construct incorporating a protein sequence able to bind albumin may have an error rate of at most 1×10⁻³, 5×10⁻⁴, ×10⁻⁴, 5×10⁻⁵, 1×10⁻⁵, 5×10⁻⁶, or less. The Taq polymerase construct incorporating a protein sequence able to bind albumin may have an error rate of at least 1×10⁻³, 5×10⁻⁴, 1×10⁻⁴, 5×10⁻⁵, 1×10⁻⁵, 5×10⁻⁶, or more.

The polymerase (e.g., Taq-polymerase) construct that incorporates a protein sequence able to bind albumin can be about 95%, 96%, 97%, 98%, 99%, 99.5%, 99.9%, 99.99% pure. The polymerase (e.g., Taq-polymerase) construct that incorporates a protein sequence able to bind albumin may not yield nonspecific amplification during an amplification reaction. The nonspecific amplification may not be observed after 10, 15, 20, 25, 30, 35, 40, 45, 50, 55, 60, 65, 70, 80, 90, 100 cycles or more in a reaction. The polymerase (e.g., Taq-polymerase) construct that incorporates a protein sequence able to bind albumin may have a nucleic acid contaminant less than about 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or less.

The invention may comprise Taq polymerase buffer, which may improve GC rich regime amplification. Such Taq Polymerase buffer may be applied in single cell analysis and next generation sequencing. The buffer components can be adjusted to increase amplification efficiency. For example, the buffer components comprise MgCl₂, KCl, Tris-HCl, Tween 20, and BSA. The buffer components may comprise (alkali earth) (halogen)₂, (alkali) (halogen), Tris-HCl, Tween 20, and BSA. The monovalent halogen anion can be fluorine (F), chlorine (Cl), bromine (Br), iodine (I), astatine (At) or any combination thereof. The alkali monovalent cation can be lithium (Li), sodium (Na), potassium (K), rubidium (Rb), caesium (Cs), francium (Fr), or any combination thereof. The alkali earth bivalent cation may be beryllium (Be), magnesium (Mg), calcium (Ca), strontium (Sr), barium (Ba), radium (Ra), or any combination thereof. One or more of the components can be adjusted to increase amplification efficiency. The concentrations of MgCl₂, KCl, Tris-HCl, Tween 20, and BSA or HSA can be increased or decreased. The concentrations of MgCl₂ and BSA or HSA can be increased, or decreased, to increase amplification efficiency. The concentrations of (alkali earth)(halogen)₂, (alkali)(halogen), Tris-HCl, Tween 20, and BSA or HSA can be increased or decreased. The concentrations of (alkali earth)(halogen)₂ and BSA can be increased, or decreased, to increase amplification efficiency.

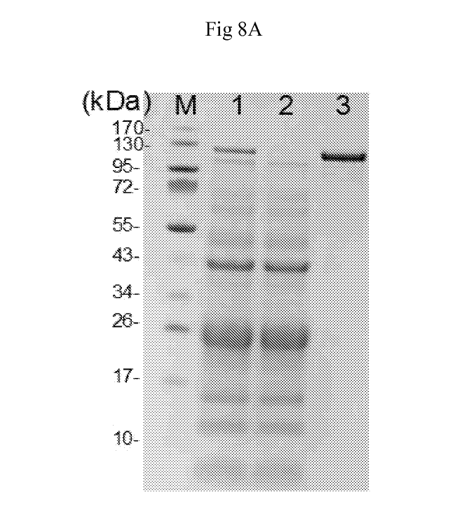

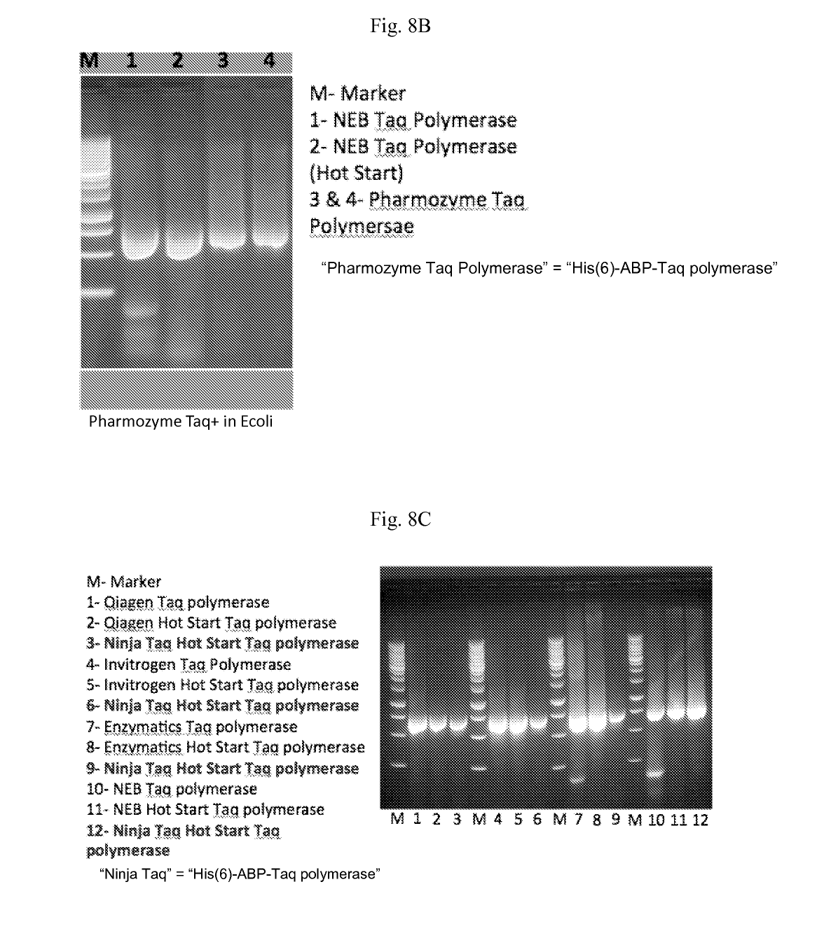

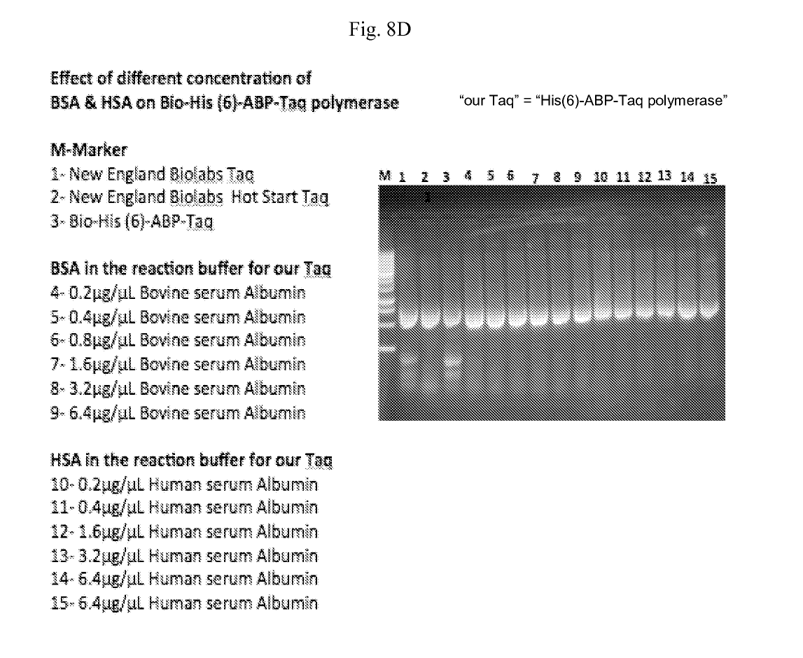

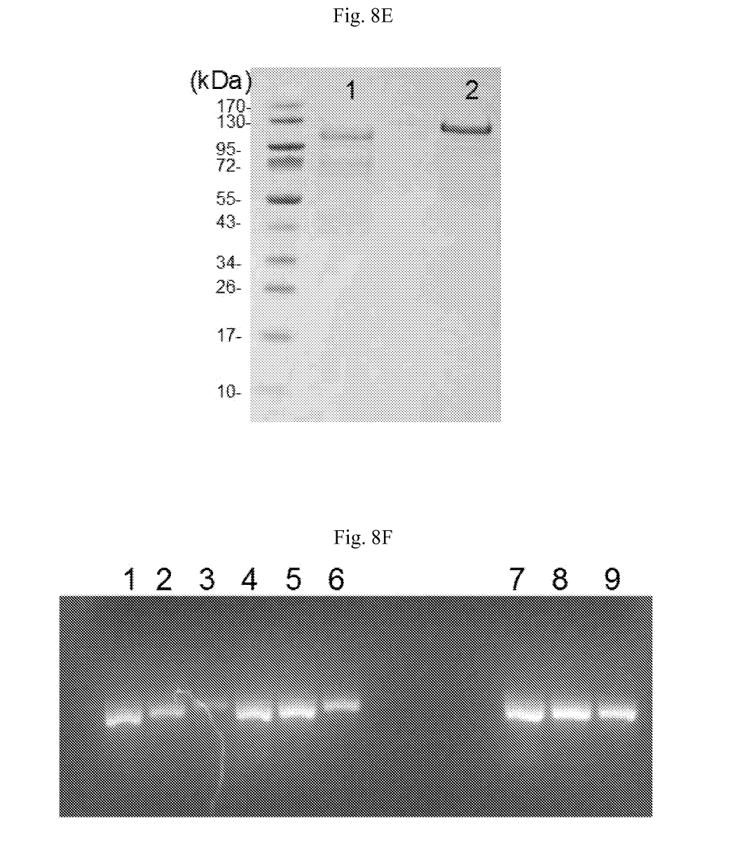

The invention may comprise a detection assay such as a polynucleotide amplification methodology to evaluate the activity of a polymerase, such as a modified polymerase described here. Sometimes, the amplification methodology comprises Real Time PCR. Occasionally, the amplified polynucleotide will have an increased sensitivity relative to commercially available polynucleotide amplification kids (e.g., see FIG. 8C). Such increased sensitivity may be an increase of at least 2, 3, 4, 5, 6, 7, 8, 9, 10, 15, 20, 25, 30, 35, 40, 45, 50, 55, 60, 65, 70, 75, 80, 85, 90, 95, 100, 200, 500, or more in sensitivity.

At times, the invention comprises high sensitivity real time genetic sequencing machine, this sequencing ability may comprise integrated hardware and chemistry technology for high sensitivity genetic amplification analysis (such as Real Time PCR). In one example, the sequencing methodology may include optical sensing of colors (i.e. chromophores), fluorescence or phosphorescence. Sometimes, the sequencing methodology may include sensing of bioluminescence.

In some examples, the invention comprises apparatus, systems, methods and kits for the evaluation of the activity of a polymerase. As described elsewhere herein, the kit may comprise reagents and buffers for polynucleotide (e.g. DNA) amplification, additional enzymes to further facilitate the amplification process, or to allow quantification of the amplification process. Sometimes, the kit may further comprise specially designed oligonucleotides such as the oligonucleotide of Formula (I) and respective primers to facilitate the evaluation of the activity of a polymerase.

The amplification methodology may comprise a polymerase construct incorporating a protein sequence able to bind albumin which may or may not require anti-polymerase antibody such as anti-Taq polymerase antibody as is used in the art. The albumin can be serum albumin. The albumin may be mammalian albumin (e.g. human or bovine albumin). Anti-Taq polymerase antibody may keep the Taq DNA polymerase from being activated at storage conditions, (e.g. lower temperature) prevents nonspecific amplification and primer-dimer formation during PCR amplification. Anti-Taq polymerase may include anti-Taq polymerase monoclonal antibodies from eENZYME LLC, BIORON, GeneON, or TOYOBO, and AccuStart™ Taq antibody (Quanta BioSciences).

The polymerase (e.g. Taq polymerase) construct incorporating a protein sequence able to bind albumin may be used with any suitable polynucleotide sequencing techniques. Such sequencing techniques may comprise conventional sequencing methodologies such as Sanger sequencing, Illumina (Solexa) sequencing, pyrosequencing, next generation sequencing, Maxam-Gilbert sequencing, chain termination methods, shotgun sequencing, bridge PCR. Next generation sequencing methodologies may comprise Massively parallel signature sequencing, Polony sequencing, SOLiD sequencing, Ion Torrent semiconductor sequencing, DNA nanoball sequencing, Heliscope single molecule sequencing, Single molecule real time (SMRT) sequencing. Other sequencing methodologies that may be used comprise Nanopore DNA sequencing, Tunnelling currents DNA sequencing, Sequencing by hybridization, Sequencing with mass spectrometry, Microfluidic Sanger sequencing, Microscopy-based techniques, RNA Polymerase sequencing, In vitro virus high-throughput sequencing, or any other sequencing methodologies used in the art.

The methodology may further comprise production of DNA ligases such as T4 ligases, any mammalian ligase such as DNA ligase I, DNA ligase III, DNA ligase IV, eukaryotic DNA ligase, thermostable ligase, or any other ligase known in the art.

The amplification methodologies can be used to amplify the polynucleotides. Polynucleotide amplification may include any amplification such as polymerase chain reaction (PCR), nucleic acid sequence based amplification (NASBA), self-sustained sequence replication (3SR), loop mediated isothermal amplification (LAMP), strand displacement amplification (SDA), whole genome amplification, multiple displacement amplification, strand displacement amplification, helicase dependent amplification, nicking enzyme amplification reaction, recombinant polymerase amplification, reverse transcription PCR (RT-PCR), ligation mediated PCR, methylation specific PCR, digital PCR, hot start PCR, multiplex ligation-dependent probe amplification (MLPA), multiplex-PCR, nested PCR, overlap-extension PCR (also as splicing by overlap extension or SOEing), quantitative PCR (qPCR), or any other amplification known in the art. Whole Genome Amplification Applications may include IVF, CTC Cancer Detection, single cell research, Stem Cell Research require sample preservation or clean amplification. In some examples of the invention, amplification methodologies used herein include amplification reactions that release pyrophosphate during the amplification process of a polynucleotide strand.

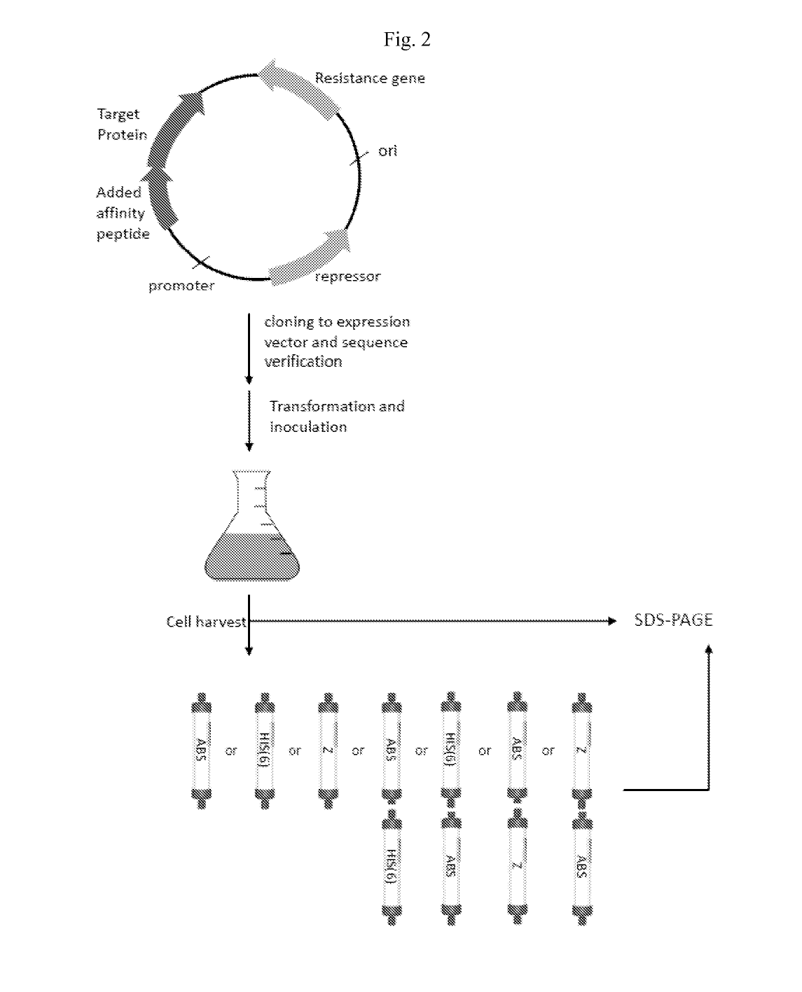

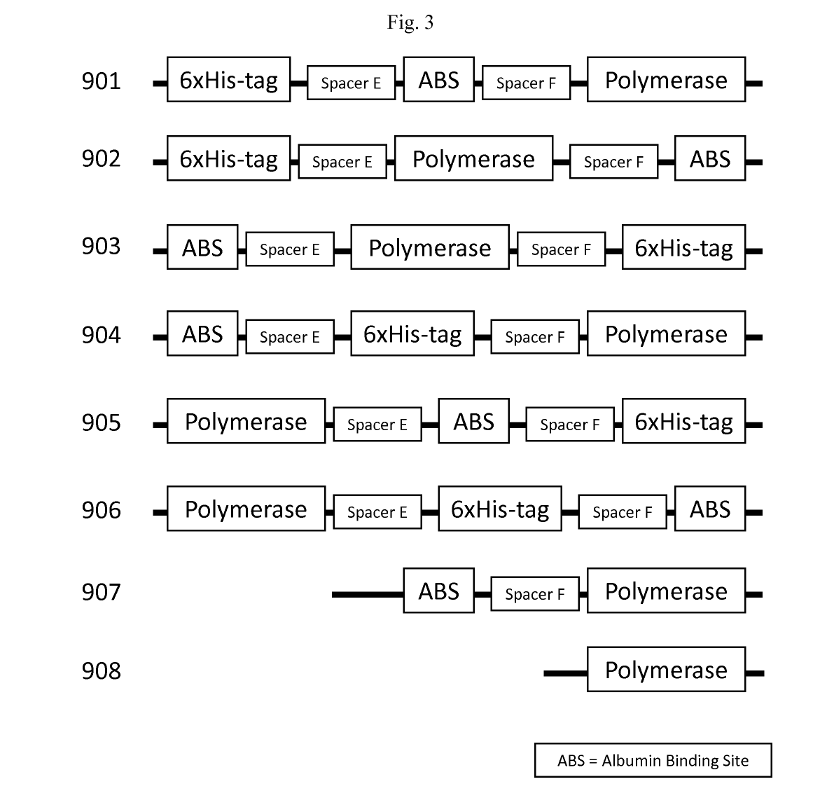

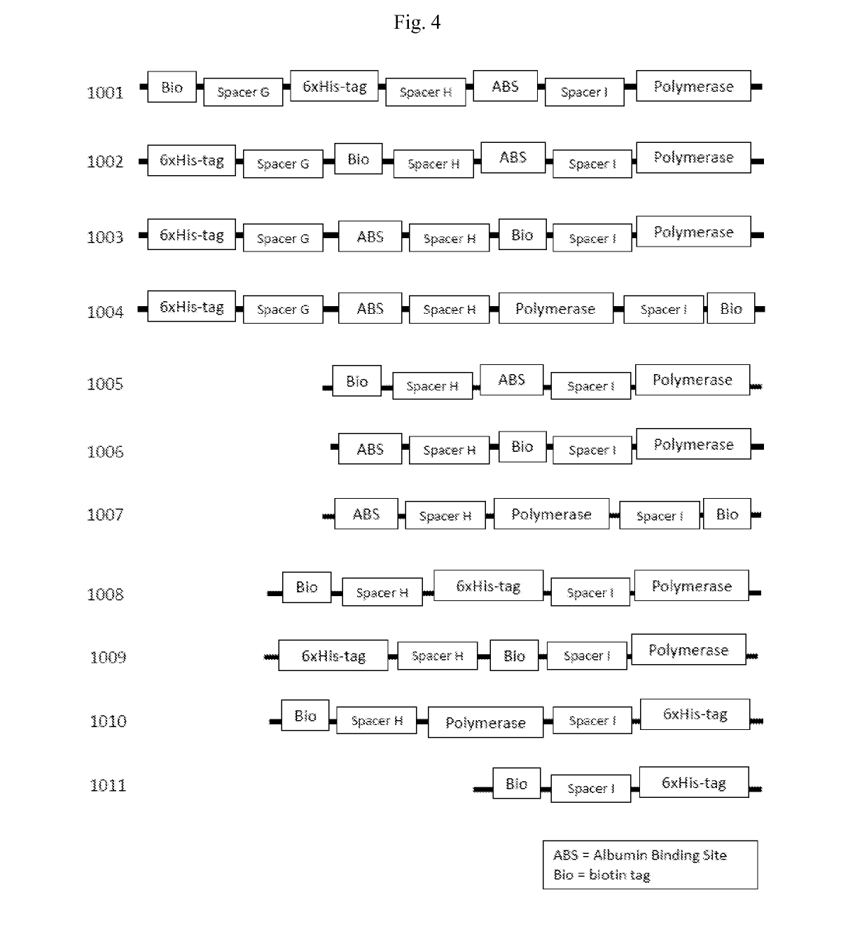

In some aspects, described herein is a protein expression and purification methodology of various modified polymerases. The modified polymerase can be Taq polymerase. The modified polymerase can be directly or indirectly connected to one, two, three or more protein purification tags. Such purification tags preferentially bind to a specific molecular-target. Examples for tag—molecular-target pairs are antigen-antibody, enzyme-substrate or receptor-ligand. The purification tag can be an amino acid sequence. Sometimes, the modified polymerase is directly or indirectly linked to an amino acid sequence (e.g. a peptide) that is able to bind a specific moiety (e.g. protein, or molecular-target). Such amino acid sequence is referred to as an affinity amino acid sequence (e.g. affinity peptide) or purification tag (e.g. protein purification tag). Such amino acid sequence may have a high binding affinity to a specific protein or a specific molecular-target. Such preferred binding affinity can be manipulated by variation of external conditions that are at least immediately adjacent to the complex formed between the affinity amino acid sequence and the specific protein. Such external conditions may include temperature, pH, conductivity, salt concentration, or other external stimuli. The binding between the modified polymerase and the affinity amino acid sequence (i.e. purification tag) may be covalent. The covalent binding may be direct or through a molecular spacer. Examples for affinity-amino-acid and specific protein/molecular-target pairs are albumin-binding-protein and albumin; hexa-histidine (also referred to as his-tag, histidine-tag, HIS6-tag, 6× his-tag, HIS(6)) and bivalent cobalt or nickel (forming HIS(6)-cobalt or nickel complex respectively), ABP-Z and protein A, and/or biotin-tag and biotin (FIG. 2). Sometimes, the modified polymerase is further directly or indirectly covalently linked to an amino acid sequence (e.g. peptide or protein) able to bind albumin, forming a protein construct of a target protein and an amino acid sequenceable to bind albumin. The albumin may be bovine or human albumin. The amino acid sequence that is able to bind albumin contains an albumin binding site (ABS), also known as an albumin binding domain. In some instances, the binding affinity (related to one over the dissociation constant (1/K_{d})) is of at least 0.1, 0.5, 1, 1.5, 2, 2.5, 3 or more nanomolar (nM) of albumin to an albumin binding domain. In some instances, the binding affinity is at most 0.1, 0.5, 1, 1.5, 2, 2.5, 3 or less nanomolar (nM) of albumin to an albumin binding domain. Occasionally, the binding affinity of albumin binding domain to albumin is of about 1 nanomolar (nM). The albumin may be mammalian albumin. The mammalian albumin can be bovine or human albumin. The albumin may be serum albumin (e.g. human serum albumin or bovine serum albumin). Sometimes, the modified polymerase is further directly or indirectly covalently linked to a HIS moiety, such as a six histidines (His-6), forming a protein construct of a modified polymerase and a HIS-6. In some examples, the modified polymerase is directly or indirectly covalently linked to both HIS (e.g., His-6) and ABS, forming a protein construct of a modified polymerase, a HIS (e.g., HIS-6), and ABS. Such covalent linkage of the three components (modified polymerase, ABS and HIS) may be in any order, as exemplified in FIG. 3, for specific respective examples where ABS is albumin binding protein ABP in Streptococcal Protein G, and the modified polymerase is Taq-polymerase. In some examples, the modified polymerase is directly or indirectly covalently linked to HIS (e.g., HIS-6), ABS, and biotin-tag, forming a protein construct of a modified polymerase, a HIS (e.g., HIS-6), ABS, and biotin-tag. Such covalent linkage of the four components (modified polymerase, ABS, HIS (e.g., HIS-6), and biotin-tag) may be in any order, as exemplified in FIG. 4, for specific respective examples where ABS is albumin binding protein ABP in Streptococcal Protein G, and the modified polymerase is Taq-polymerase. In some instances, ABP is a modified ABP that is able to bind protein A (i.e. ABP-Z). In some instances, ABS is further connected to an amino acid moiety able to bind protein A. In some instances, ABS is connected to both HIS (e.g., HIS-6) and to an amino acid moiety able to bind protein A. In some examples, ABS is connected to Z domain (from Staphylococcal protein A, SpA). The modified polymerase can be directly or indirectly covalently linked to Z domain (or Z domain moiety). The modified polymerase can be directly or indirectly covalently linked to HIS, ABS, BIO, and/or Z domain (or Z domain moiety). The modified polymerase can be directly or indirectly covalently linked to HIS, ABS, and Z domain (or Z domain moiety). The modified polymerase can directly or indirectly covalently linked to BIO, HIS, and Z domain (or Z domain moiety). Sometimes, Z domain (or Z domain moiety) can comprise Z domain wild-type, Zacid₂, or Zbasic₂. The modified polymerase can directly or indirectly covalently linked to BIO, HIS, ABS, and Z domain wild-type. The modified polymerase can directly or indirectly covalently linked to HIS, ABS, and Z domain wild-type. The modified polymerase can directly or indirectly covalently linked to BIO, ABS, and Z domain wild-type. The modified polymerase can directly or indirectly covalently linked to BIO, HIS and Z domain wild-type. The modified polymerase can directly or indirectly covalently linked to BIO, HIS, ABS, and Zacid₂. The modified polymerase can directly or indirectly covalently linked to HIS, ABS, and Zacid₂. The modified polymerase can directly or indirectly covalently linked to BIO, ABS, and Zacid₂. The modified polymerase can directly or indirectly covalently linked to BIO, HIS and Zacid₂. The modified polymerase can directly or indirectly covalently linked to BIO, HIS, ABS, and Zbasic₂. The modified polymerase can directly or indirectly covalently linked to HIS, ABS, and Zbasic₂. The modified polymerase can directly or indirectly covalently linked to BIO, ABS, and Zbasic₂. The modified polymerase can directly or indirectly covalently linked to BIO, HIS and Zbasic₂.

Described herein is a composition which comprises a polymerase with less than about 60 ng/mL, 50 ng/mL, 40 ng/mL, 30 ng/mL, 20 ng/mL, or about 10 ng/mL of nucleic acid contaminant. The polymerase can be a DNA polymerase or an RNA polymerase. The polymerase can comprise Taq polymerase, Vent polymerase, Klenow Fragment (3′-5′ exo-), DNA Polymerase I (large Klenow fragment), E. coli DNA polymerase I, phi29 DNA polymerase, Phusion DNA polymerase, or T4 DNA polymerase. The polymerase can be Taq polymerase. Taq polymerase can be native or modified Taq polymerase. Taq polymerase can further comprise an albumin binding moiety, a HIS moiety, a biotin-tag moiety, or combinations thereof. The albumin binding moiety can be directly connected to the Taq polymerase or is connected to the Taq polymerase though a spacer. A genetic sequence of the albumin binding moiety and the Taq polymerase can comprise the albumin binding moiety sequence residing on the 3′ end of the Taq polymerase sequence, residing on the 5′ end of the Taq polymerase sequence, or residing on both the 3′ end and 5′ end of the Taq polymerase sequence. The composition can further comprise an albumin. The albumin can inhibit the activity of the polymerase by binding to the polymerase at a temperature of from about 0° C. to about 60° C., from about 20° C. to about 55° C., or from about 25° C. to about 50° C. The albumin can be inactivated at a temperature of at least 61° C. or higher. The polymerase can regain its enzymatic activity at a temperature of at least 61° C. or higher. The albumin can be mammalian albumin or a mammalian albumin analogue. The albumin can be human serum albumin. The albumin can be bovine serum albumin. The albumin binding moiety able to bind serum albumin can be at least a part of Streptococcal protein G. The at least a part of Streptococcal protein G can be the entire Streptococcal protein G. The at least a part of Streptococcal protein G can comprise ABP (121aa), BB (214aa), ABD (46aa), ADB1 binding site, ADB2 binding site, or ADB3 binding site. The ABD to albumin affinity can be 1.5 nanomolar or less. The ABD to human serum albumin affinity can be 1.5 nanomolar or less. The polymerase can have the sequence as illustrated in SEQ ID NO: 1. The polymerase can be expressed in an eukaryotic cell. The eukaryotic cell can be a yeast cell. The yeast can be Pichia pastoris. The polymerase can be expressed in E. coli.

Described herein is a method for amplifying a target DNA which comprises (a) incubating the target DNA with a polymerase having the polymerase construct described herein, an albumin, a set of primers, and nucleoside phosphates selected from the group consisting of adenine, thymine, guanine, cytosine, and uridine; so as to form a reaction mixture; and (b) subjecting the reaction mixture to an amplification method, whereby the set of primers is extended by the polymerase to amplify the target DNA sequence. The albumin can inhibit the activity of the polymerase by binding to the polymerase at a temperature of from about 0° C. to about 60° C., from about 20° C. to about 55° C., or from about 25° C. to about 50° C. The albumin can be inactivated at a temperature of at least 61° C. or higher. The polymerase can regain its enzymatic activity at a temperature of at least 61° C. or higher. The polymerase can be expressed in an eukaryotic cell. The eukaryotic cell can be a yeast cell. The yeast can be Pichia pastoris. The polymerase can be expressed in E. coli. The amplification method can be a polymerase chain reaction (PCR). The amplification method can be a next-generation sequencing method.

### Taq Polymerase Construct

In some cases, disclosed herein is a polynucleotide amplification enzyme that comprises an amplification enzyme and a protein able to bind to albumin. The amplification enzyme may be a polymerase protein. Described herein is a polymerase construct comprising at least one moiety that is capable of binding albumin. The polymerase protein may be a DNA polymerase or an RNA polymerase. The polymerase protein may be a DNA polymerase. Exemplary DNA polymerases are disclosed elsewhere herein, and may include Bst DNA polymerase, Bsu DNA polymerase, Crimson Taq DAN polymerase, Deep Vent_{R}™ DNA polymerase, Deep Vent_{R}™ (exo-) DNA polymerase, E. coli DNA polymerase I, Klenow fragment (3′-5′ exo-), DNA polymerase I (large Klenow fragment), LongAmp® Taq DNA polymerase, LongAmp® Hot Start Taq DNA polymerase, M-MuLV reverse transcriptase; One Tag® DNA polymerase, One Taq® Hot Start DNA polymerase, phi29 DNA polymerase, Phusion® Hot Start Flex DNA polymerase, Phusion® High-Fidelity DNA polymerase, Q5®+Q5® Hot Start DNA polymerase, Sulfolobus DNA polymerase IV, T4 DNA polymerase, Theminator™ DNA polymerase, Vent_{R}™ DNA polymerase, and Vent_{R}™ (exo-) DNA polymerase. The polymerase can comprise Taq polymerase, Vent polymerase, Klenow Fragment (3′-5′ exo-), DNA Polymerase I (large Klenow fragment), E. coli DNA polymerase I, phi29 DNA polymerase, Phusion DNA polymerase, or T4 DNA polymerase. The polymerase protein can be a Taq polymerase. The Taq polymerase can be a native Taq polymerase or a modified Taq polymerase. In some cases, the Taq polymerase is a modified Taq polymerase. In some cases, the protein able to bind to albumin contains an albumin binding site (ABS).

The modified Taq polymerase construct may contain the modified Taq polymerase portion and an ABS portion. The modified Taq polymerase construct may further comprise a polyhistidine-tag (e.g., 6× His-tag). The modified Taq polymerase construct may further comprise a polyhistidine-tag (e.g., 6× His-tag) and ABS. In some instances, HIS (e.g., 6× His-tag) is used as a purification tag. Sometimes, ABP is used as a purification tag. In some cases, ABS is incorporated in an immunoglobulin-binding protein. ABS may be incorporated in Protein G such as in Streptococcal Protein G. ABS may incorporate albumin binding protein (ABP). At times, ABS is ABP, such as ABP from Streptococcal Protein G. In some instances, a fusion Taq polymerase protein is referred to as HIS-ABS-Taq polymerase. The HIS (e.g., HIS-6) tag, ABS, and Taq polymerase may be directly connected to each other. The HIS (e.g., HIS-6) tag, ABS, and Taq polymerase may be connected through spacers. Exemplary HIS-ABS-Taq polymerase constructs are shown in FIG. 3. In some cases, the HIS-ABS-Taq polymerase construct is the construct shown as 901, 902, 903, 904, 905, or 906. The HIS-ABS-Taq polymerase construct may be the construct shown as 901. The HIS-ABS-Taq polymerase construct may further be modified to remove the HIS (e.g., HIS-6) tag portion, shown as 907. The HIS (e.g., HIS-6) tag portion may be cleaved off during or post purification process. Spacer E may contain an enzyme cleavage site (e.g., protease cleavage site), which allows removal of the HIS (e.g., HIS-6) tag from the ABS-Taq polymerase portion. The HIS-ABS-Taq polymerase construct may be further modified to remove both the 6× His-tag portion and the ABS portion, shown as 908. Spacer F may also contain an enzyme cleavage site (e.g., protease cleavage site). At times, the enzyme cleavage sites in spacer E and spacer F are the same. The enzyme cleavage sites in spacer E and spacer F may be different.

The modified Taq polymerase construct may contain the modified Taq polymerase portion, an ABS portion, a HIS (e.g., HIS-6) tag, and further comprise a biotin-tag (Bio). In some instances, a fusion Taq polymerase protein is referred to as Bio-HIS-ABS-Taq polymerase. The biotin-tag (Bio), HIS (e.g., HIS-6) tag, ABS, and Taq polymerase may be directly connected to each other. The biotin-tag (Bio), HIS (e.g., HIS-6) tag, ABS, and Taq polymerase may be connected through spacers. Exemplary Bio-HIS-ABS-Taq polymerase constructs are shown in FIG. 4. In some cases, the Bio-HIS-ABS-Taq polymerase construct is the construct shown as 1001, 1002, 1003, 1004, 1005, 1006, 1007, 1008, 1009, 1010, or 1011. The Bio-HIS-ABS-Taq polymerase construct may be the construct shown as 1001. The Bio-HIS-ABS-Taq polymerase construct may further be modified to remove the HIS (e.g., HIS-6) tag portion, shown as 1005, 1006, or 1007. The Bio-HIS-ABS-Taq polymerase construct may further be modified to remove the ABS portion, shown as 1008, 1009, or 1010. The Bio-HIS-ABS-Taq polymerase construct may be further modified to remove both the HIS (e.g., HIS-6) tag portion and the ABS portion, shown as 1011. Spacers G, H and I may each contain an enzyme cleavage site (e.g., protease cleavage site). At times, the enzyme cleavage sites in spacer G, spacer H, and spacer I may be the same. The enzyme cleavage sites in spacer G, spacer H, and spacer I may be different.

The distance between either the ABS, the HIS or both, and the modified Taq polymerase protein is defined by spacer E and spacer F. Both spacer E and spacer F represent molecule linkage such as covalent linkage. Spacer E may be at least 1, 2, 3, 4, 5, 6, 7, 8, 9, 10, or more covalently linked amino acids. Spacer F may be at least 1, 2, 3, 4, 5, 6, 7, 8, 9, 10, or more covalently linked amino acids. The number of covalently linked amino acids in spacer E may be different than the number of covalently linked amino acids in spacer F. The number of covalently linked amino acids in spacer E may be the same as the number of covalently linked amino acids in spacer F. The spacer molecule may be a chimeric peptide, an organic molecule, saccharide, a peptide, a polynucleotide or a nucleic acid monomer. The organic molecule may be aliphatic, conjugated or aromatic. The conjugated organic molecule may comprise at least 1, 2, 3, 4, 5, 6, 7, 8, 9, 10 or more conjugated bonds. The saccharide may be a mono, di, oligo or polysaccharide. In some examples spacer E is identical to spacer F. In some instances, spacer E is different than spacer F.

The distance between either the biotin-tag (Bio), ABS, the HIS (e.g., HIS-6), and the modified Taq polymerase protein is defined by spacer G, spacer H, and spacer I. Spacer G, spacer H, and spacer I represent molecule linkage such as covalent linkage. Spacer G may be at least 1, 2, 3, 4, 5, 6, 7, 8, 9, 10, or more covalently linked amino acids. Spacer H may be at least 1, 2, 3, 4, 5, 6, 7, 8, 9, 10, or more covalently linked amino acids. Spacer I may be at least 1, 2, 3, 4, 5, 6, 7, 8, 9, 10, or more covalently linked amino acids. The number of covalently linked amino acids in spacer G may be different than the number of covalently linked amino acids in spacer H and/or spacer I. The number of covalently linked amino acids in spacer G may be the same as the number of covalently linked amino acids in spacer H and/or spacer I. The spacer molecule may be a chimeric peptide, an organic molecule, saccharide, a peptide, a polynucleotide or a nucleic acid monomer. The organic molecule may be aliphatic, conjugated or aromatic. The conjugated organic molecule may comprise at least 1, 2, 3, 4, 5, 6, 7, 8, 9, 10 or more conjugated bonds. The saccharide may be a mono, di, oligo or polysaccharide. In some examples spacer G is identical to spacer H and/or spacer I. In some instances, spacer G is different than spacer H and/or spacer I.

As disclosed above, in some instances ABS is obtained from Protein G. Protein G is an immunoglobulin-binding protein that serves as a bacterial receptor on the surface of Gram-positive bacteria. In some instances, Protein G is expressed in group C and group G of Streptococcal bacteria. ABP may be obtained from Streptococcal protein G (SpG), strain G148.

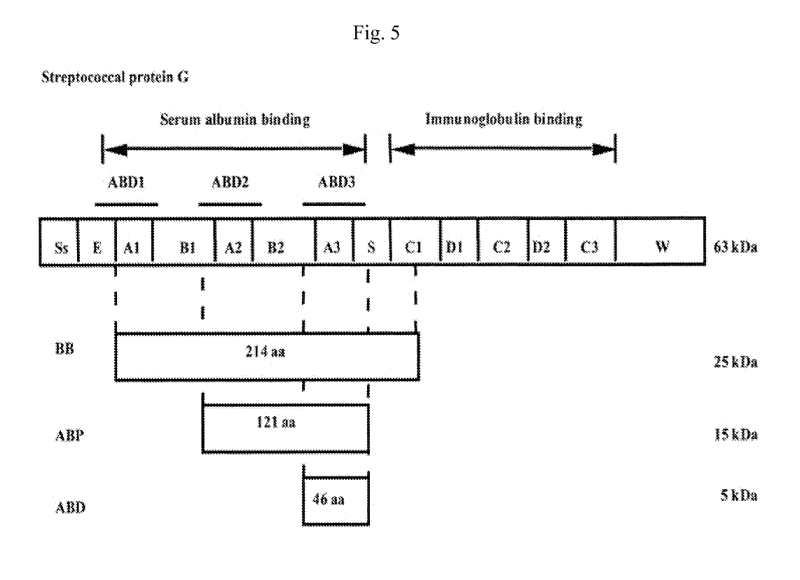

Any portions of the Protein G protein containing ABS may be connected to the modified Taq polymerase (see FIG. 5). In some cases, the ABS is ABP. Sometimes, ABS is at least one of ABD1, ABD2, and ABD3 regions of the Protein G. BB region may be connected to Taq polymerase. ABP region may be connected to Taq polymerase. ABD region may be connected to Taq polymerase. In some cases, the entire Streptococcal protein G is connected to Taq polymerase. In some instances, the HIS (e.g., His(6)) moiety is further connected to the modified Taq polymerase construct with ABS. The HIS (e.g., His(6)) moiety may be connected to Taq polymerase (such construct lacks an albumin binding site).

In some instances, the ABS further comprises a second binding site. The second binding site may be within the ABP. The second binding site may be linked to the ABP, either through direct covalent linkage or non-directly (e.g. a spacer). The second binding site may be at a site different from the ABP binding site and does not interfere with the interaction of ABP with albumin. The second binding site may be a binding site that recognizes a domain of a membrane protein. The membrane protein can be a type I membrane protein from a bacterium. The membrane protein can be a Staphylococcal protein A (SpA). The second binding site can recognize one or more domains of SpA. Sometimes, the second binding site recognizes domain B of SpA. Occasionally, the second binding site recognizes an analog of domain B, Z domain. In some cases, the second binding site is Z domain binding site. In some instances, the ABP further comprises the Z domain binding site, referred herein as ABP-Z. At most 1, 2, 5, 8, 10, 11, 12, or 15 amino acid residues on ABP can be genetically modified to generate the second binding site. At most 1, 2, 5, 8, 10, 11, 12, or 15 amino acid residues on ABP can be genetically modified to generate the second binding site that recognizes Z domain. Sometimes, about 11 amino acid residues are genetically modified in ABP. In some cases, amino acid residues corresponding to residue position 22, 25, 26, 29, 30, 33, 34, 54, 57, 58 and 62 are genetically modified to generate the second binding site that recognizes the Z domain. Genetic modifications such as site-specific modification are described elsewhere herein. The ABS construct may be produced using the general procedure for production of the target protein construct. In some cases, the ABS construct is described in FIG. 2.

The modified Taq polymerase construct can comprise Z domain (or Z domain moiety) from SpA in combination with HIS, ABS, and/or biotin-tag (e.g., biotin binding domain tag). The Z domain (or Z domain moiety), HIS, ABS, and/or biotin-tag can be in any order and can be genetically introduced at the 5′ terminal of the Taq polymerase sequence, 3′ terminal of the Taq polymerase sequence, or at both termini of the Taq polymerase sequence. The Z domain (or Z domain moiety), HIS, ABS, and/or biotin-tag can comprise one or more spacers between each individual component. For example, a spacer can be introduced between Z domain (or Z domain moiety) and HIS, or a first spacer can be introduced between Z domain (or Z domain moiety) and HIS and a second spacer can be introduced between HIS and ABS, and so forth. Sometimes, Z domain (or Z domain moiety), HIS, ABS, and/or biotin-tag can be covalently linked without spacers between each individual component. Sometimes, Taq polymerase, Z domain (or Z domain moiety), HIS, ABS, and/or biotin-tag can be in any order within the modified Taq polymerase construct. Sometimes, Z domain (or Z domain moiety) can replace ABS in the modified Taq polymerase construct. The Taq polymerase, Z domain (or Z domain moiety), HIS, and BIO can comprise one or more spacers between each individual component and Taq polymerase, Z domain, HIS, and BIO can be in any order. The Taq polymerase, Z domain (or Z domain moiety), HIS, and BIO may not comprise one or more spacers between each individual component and Taq polymerase, Z domain (or Z domain moiety), HIS, and BIO can be in any order.

Sometimes, the Z domain (or Z domain moiety) is referred to as Z domain wild type, Zacid₂, or Zbasic₂ (see FIG. 10). The Z domain (or Z domain moiety) can have the sequences VDNKFNKEQQNAFYEILHLPNLNEEQRNAFIQSLKDDPSQ SANLLAEAKKLNDAQPK (SEQ ID NO: 11) (Z domain wild-type), VDNKFNKEEEEAEEEIEELPNLNEEQEEAFIESLEDDPSQSANLLAEAKKLNDAQPK (SEQ ID NO: 12) (sometimes can be referred to as Zacid₂), or VDNKFNKERRRARREIRHLPNLNEEQRRAFIRSLRDDPSQSANLLAEAKKLNDAQPK (SEQ ID NO: 13) (sometimes can be referred to as Zbasic₂). The Z domain (or Z domain moiety) can have the sequence VDNKFNKEQQNAFYEILHLPNLNEEQRNAFIQSLKDDPSQSANLLAEAKKLNDAQPK (SEQ ID NO: 11) (Z domain wild-type). The Z domain (or Z domain moiety) can have the sequence VDNKFNKEEEEAEEEIEELPNLNEEQEEAFIESLEDDPSQSANLLAEAKKLNDAQPK (SEQ ID NO: 12) (sometimes can be referred to as Zacid₂). The Z domain (or Z domain moiety) can have the sequence VDNKFNKERRRARREIRHLPNLNEEQRRAFIRSLRDDPSQSANLLAEAKKLNDAQPK (SEQ ID NO: 13) (sometimes can be referred to as Zbasic₂).

The modified Taq polymerase can comprise the sequence of SEQ ID NO: 1. The modified Taq polymerase can consist of the sequence of SEQ ID NO: 1.

The modified Taq polymerase can be generated through any suitable mutagenesis methods. In some instances, the modified Taq polymerase is generated through a site-directed mutagenesis method. As disclosed above, site-directed mutagenesis is a method that allows specific alterations or modifications within the gene of interest. The site-directed mutagenesis can utilize Cassette mutagenesis method, PCR-site-directed mutagenesis, whole plasmid mutagenesis, Kunkel's method, or in vivo site-directed mutagenesis method. The modified Taq polymerase can be generated through random mutagenesis method. Random mutagenesis is a method of generating a library of protein mutants with different functional properties. Random mutagenesis can be achieved using error-prone PCR approach, rolling circle error-prone PCR approach, mutator strains approach, temporary mutator strains approach, insertion mutagenesis approach, ethyl methanesulfonate approach, the nitrous acid approach, or DNA shuffling. In some instances, random mutagenesis utilizing an error-prone PCR approach is used to generate a modified Taq polymerase.

Amplification enzyme and the albumin construct may be thermostable. The amplification enzyme may be a polymerase protein. The polymerase protein may be a DNA polymerase. Exemplary DNA polymerases are described elsewhere herein, and may include Bst DNA polymerase, Bsu DNA polymerase, Crimson Taq DAN polymerase, Deep Vent_{R}™ DNA polymerase, Deep Vent_{R}™ (exo-) DNA polymerase, E. coli DNA polymerase I, Klenow fragment (3′-5′ exo-), DNA polymerase I (large Klenow fragment), LongAmp® Taq DNA polymerase, LongAmp® Hot Start Taq DNA polymerase, M-MuLV reverse transcriptase; One Taq® DNA polymerase, One Taq® Hot Start DNA polymerase, phi29 DNA polymerase, Phusion® Hot Start Flex DNA polymerase, Phusion® High-Fidelity DNA polymerase, Q5+Q5® Hot Start DNA polymerase, Sulfolobus DNA polymerase IV, T4 DNA polymerase, Theminator™ DNA polymerase, Vent_{R}™ DNA polymerase, and Vent_{R}™ (exo-) DNA polymerase. The DNA polymerase may be a Taq polymerase. The amplification enzyme and the albumin construct may be the Taq polymerase and albumin construct. The Taq polymerase and albumin construct may be thermostable. The thermo stable Taq-polymerase and albumin construct may be stable in temperature of at least 50, 51, 52, 53, 54, 55 or more degrees Celsius.

In some instances, dNTPs are incorporated in an iterative manner onto the DNAₜₑₘₚₗₐₜₑ by a polynucleotide (e.g. DNA) polymerase such as Taq polymerase. The polymerase may be an enzyme construct that incorporates a protein able to bind albumin, for example a Taq polymerase construct with ABS. The polymerase may be a Taq polymerase construct capable of binding to albumin. In some instances the albumin is human or bovine albumin. Albumin may be serum albumin (e.g. human serum albumin or bovine serum albumin). The albumin may be any albumin type, for example bovine albumin or human albumin. The albumin may be serum albumin. Sometimes, the introduction of each type of dNTP is controlled. The dNTP type may be introduced one by one into the reaction mixture.

The Taq polymerase construct can be produced using the production procedure described infra. Sometimes, the Taq polymerase construct is produced using a bioengineered host as mentioned above. In some cases, purification of the modified Taq polymerase protein can utilize the purification procedure described infra. The purification scheme may be the scheme illustrated in FIG. 2. Sometimes, the modified Taq polymerase further undergoes a nucleic acid de-contamination step. Sometimes, the activity of the modified Taq polymerase may be evaluated based on a polymerase activity characterization assay described herein.

### Procedure for Production of Taq Polymerase Construct

In some aspects, any of the above-mentioned Taq protein constructs are produced using vector transformation. Sometimes, the above-mentioned Taq protein constructs are produced by covalently linking individual polynucleotide (e.g. DNA) sequences encoding for protein segments comprising the Taq protein construct. Any of the above-mentioned Taq protein constructs can be produced using a protein synthesizer. Any of the above-mentioned Taq protein constructs can be fabricated using any combination of the methods mentioned in this paragraph.

In some instances, any of the above-mentioned Taq protein constructs (e.g., the construct of SEQ ID NO: 2) are produced by a bioengineered host. Such bioengineering may be effectuated by transforming the host with a suitable vector that carries the desired polynucleotide sequence of the target protein. The vector transformation can cause the target polynucleotide to be expressed into target protein by using the protein expression mechanism of the host. The vector construct can be engineered such that when the sequence carried by the vector is expressed into a protein, the protein would correspond to the target protein—ABS construct which are covalently linked. The covalent linkage may be direct or indirect (e.g. though a spacer). Sometimes the modified polymerase is covalently linked to HIS (e.g., HIS-6). The covalent linkage may be direct or indirect (e.g. though a spacer). Sometimes the modified polymerase is covalently linked to both ABS and HIS (e.g., HIS-6). Other times, the modified polymerase is covalently linked to ABS and biotin-tag (Bio), HIS (e.g., HIS-6) and biotin-tag, ABS, biotin-tag, and HIS (e.g., HIS-6), or ABS, HIS (e.g., HIS-6), and Z domain. The covalent linkage may be direct or indirect (e.g. though a spacer) in any order. See for example FIGS. 2, 3, and 4 where ABS is ABP or ABP-Z, and the modified polymerase is Taq-polymerase respectively. The ABP-Z protein may be an albumin binding protein that further contains a second binding site. The second binding site may recognize a Z domain from SpA. Exemplary vectors include, but are not limited to, pACYC 177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO 815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, and pYES3/CT yeast vector. In some instances, the vector is vectors pET21 from E.coli. Sometimes, the vector is pPIC9 from Pichia pastoris. In some examples, any of the above-mentioned Taq protein constructs is synthetically produced.

### Procedure for Purification of Taq Polymerase Construct

Purification of the modified Taq polymerase protein (e.g., Taq polymerase of SEQ ID NO: 1) is conducted using the purification method described herein. In some instances, a protein purification method is described in FIG. 2. The protein purification method comprises a native, unmodified or modified polymerase. The purification method further utilizes a HIS (e.g., HIS-6) tag, an ABS tag, a biotin-tag, or a combination thereof. Purification can be carried out using either the HIS (e.g., HIS-6) tag, the ABS tag, biotin-tag, or can be carried out in tandem with one or more purifications of the polymerase protein using the HIS (e.g., HIS-6) tag, followed by one or more purifications using the ABS tag, and/or followed by one or more purifications using the biotin-tag. In some instance, purification is carried out in tandem with one or more purifications of the polymerase protein using the ABS tag, followed by one or more purifications using the HIS (e.g., HIS-6) tag, and/or followed by one or more purifications using the biotin-tag. In some instance, purification is carried out in tandem with one or more purifications of the polymerase protein using the biotin-tag, followed by one or more purifications using the HIS (e.g., HIS-6) tag, and/or followed by one or more purifications using the ABS tag. In some cases, the purification of the ABS tag involves using an albumin-affinity column or a HIS (e.g., HIS-6) column. The purification of the ABS tag may involve using an albumin-affinity column and a HIS (e.g., HIS-6) column. The albumin-affinity column can either precede or supersede the HIS (e.g., HIS-6) column as exemplified in FIG. 2. The ABS tag may contain ABP and ABP-Z. Sometimes, the ABS tag contains ABP. In some cases, the purification of the ABS tag involves using an albumin-affinity column, or an albumin-affinity column and a Z-affinity column that utilizes ABP-Z (e.g. sites on ABS that recognizes albumin and Z-domain). The albumin-affinity column can either precede or supersede the Z-affinity column as exemplified in FIG. 2. As used herein, FIG. 2 refers to the albumin-affinity column, the HIS (e.g., HIS-6) affinity column, and the Z-affinity column as albumin column, HIS (e.g., HIS-6) column, and Z column.

In some cases, purification with the HIS (e.g., HIS-6) tag is carried out in batch mode (e.g. the use of Nickel or Cobalt-charged resin in a solution of target protein lysate) or via a column (either by gravitation filtration or by a chromatography system). Exemplary Nickel and Cobalt beads include, but are not limited to, Ni-NTA agarose (Qiagen), Ni-NTA magnetic agarose beads (Qiagen), His60 Ni Superflow Resin (Clontech Laboratories), complete His-Tag purification resin (Roche), Dynabeads® His-Tag Isolation and Pulldown cobalt beads (Life Technologies), Dynabeads® TALON® cobalt beads (Life Technologies), or HisPur Cobalt resin (Thermo Scientific). Exemplary Nickel and Cobalt containing columns include, but are not limited to, in-house packed Nickel or Cobalt columns, HiTrap® Ni-NTA columns (Qiagen), Ni-NTA Superflow columns (Qiagen), Ni-NTA Spin columns (Qiagen), His60 Ni Superflow columns (Clontech Laboratories), or HisPur Cobalt Spin Column (Thermo Scientific).

In general, the polymerase protein lysate is bound to either the Nickel-charged or Cobalt charged beads in a binding buffer containing a low concentration of imidazole. Imidazole competes with the HIS (e.g., HIS-6) tag in binding to the Nickel or Cobalt-charged beads. In some cases, the concentration of the imidazole used in the binding buffer is at most 0.01, 5, 10, 15, 20, 25, 30 millimolar (mM), or less. The concentration of the imidazole used in the binding buffer can be at least 0.01, 5, 10, 15, 20, 25, 30 millimolar (mM), or more. After the initial binding step, the beads containing the polymerase protein are subsequently washed to remove any unbound proteins. Upon completion of the washing step, the polymerase protein is then eluded using an elution buffer containing a higher concentration of imidazole than used in the binding buffer. The concentration of imidazole used in the elution buffer can be at least 200, 250, 300, 350, 400, 450, 500, 550, 600, 700, 800, 900, 1000 millimolar (mM), or more.

The eluted polymerase protein is further subjected, in some cases, to a desalting step to remove the concentration of imidazole present in the buffer. The eluted polymerase protein can be dialyzed to remove the imidazole prior to loading onto a medium containing albumin. The albumin can be mammalian albumin (e.g. bovine or human). The albumin may be serum albumin (e.g. bovine serum albumin or human serum albumin). The medium containing albumin may be particles containing bound or unbound albumin. The particles may be magnetic particles. In some instances, the particles may contain a tag. The tag can be an optical tag (e.g. a fluorescence or phosphorescence tag). The medium containing albumin may be a solid support containing bound or unbound albumin. In some instances unbound albumin is diffused into the medium. The bound albumin may be a covalently bound albumin. The medium containing albumin may be a chromatography column, which is comprised of particles having bound albumin. The albumin can be mammalian albumin (e.g. bovine or human albumin). The albumin may be serum albumin (e.g. bovine serum albumin or human serum albumin). The medium containing albumin can be a solution comprising albumin. The medium containing albumin can be a filter comprising albumin. In some instances, the filter may be a dialysis filter.

In some instances, the ABS peptide portion that is bound to the albumin is released by a change of the environment that is at least immediately adjacent to the ABS-albumin pair. The environmental change can be altering the temperature, hydrophobicity, ionic strength, conductivity or pH of the environment. A change of ionic strength can take place by alteration or addition of a salt. A change of pH may be effectuated by an addition of a base. A change of pH may be effectuated by an addition of an acid. A change of hydrophobicity may be effectuated by addition of a hydrophobic substance. The hydrophobic substance may be an alcohol such as ethanol, propanol, butanol, pentanol, hexanol, heptanol, octanol, nonanol or any combination thereof. The alcohol may comprise a linear or branched aliphatic moiety. The alcohol may comprise at least one aromatic moiety.

Separation of the ABS peptide portion from the albumin may take place by washing, immersing, or eluting the complex of ABS and albumin with a solution. The solution may be a buffer solution.

The ABS purification step may be performed on a liquid chromatography system (e.g. HPLC or FPLC) using a column immobilized with albumin. The column may be immobilized with bovine or human serum albumin. The column may be immobilized with human serum albumin. The target protein may be loaded onto a column immobilized with albumin with a loading buffer containing a neutral pH. The loading buffer may contain a basic pH (e.g. about pH 7.5, pH 8.0, or pH 8.5). The column may subsequently be washed with a washing buffer containing an acidic pH. The target protein may be eluted using an elution buffer containing an acid, such as an acetic acid, at a pH of at most 1.0, 1.5, 2.0, 2.1, 2.2, 2.3, 2.4, 2.5, 2.6, 2.7, 2.8, 2.9, 3.0, 3.5, 4.0, 4.5, 5.0, 5.5, or less. Sometimes, the pH of the elution buffer is at least 1.0, 1.5, 2.0, 2.1, 2.2, 2.3, 2.4, 2.5, 2.6, 2.7, 2.8, 2.9, 3.0, 3.5, 4.0, 4.5, 5.0, 5.5, or more. In some cases, the concentration of acid used in the elution buffer is at least 200, 250, 300, 350, 400, 450, 500, 550, 600, 700, 800, 900, 1000 millimolar (mM), or more. The eluted polymerase protein may be dialyzed into a buffer containing a neutral pH. The eluted polymerase protein may be dialyzed into a buffer containing a basic pH (e.g. about pH 7.5, pH 8.0, or pH 8.5).

In some instances, the ABS protein further comprises a Z domain recognition site (e.g. ABP-Z). The ABP-Z purification step can be performed on a liquid chromatography system (e.g. HPLC or FPLC) using a column immobilized with protein A-derived ligand. Rhe protein A-derived ligand can be alkali-tolerant. The polymerase protein can be loaded onto a column immobilized with an alkali-tolerant protein A-derived ligand with a loading buffer containing a neutral pH. The loading buffer can contain a basic pH (e.g. about pH 7.5, pH 8.0, or pH 8.5). The column can be subsequently washed with the loading buffer. The target protein can be eluted using an elution buffer containing an acid, such as an acetic acid, at a pH of at most 1.0, 1.5, 2.0, 2.1, 2.2, 2.3, 2.4, 2.5, 2.6, 2.7, 2.8, 2.9, 3.0, 3.5, 4.0, 4.5, 5.0, 5.5, or less. The pH of the elution buffer can be at least 1.0, 1.5, 2.0, 2.1, 2.2, 2.3, 2.4, 2.5, 2.6, 2.7, 2.8, 2.9, 3.0, 3.5, 4.0, 4.5, 5.0, 5.5, or more. The concentration of acid used in the elution buffer can be at least 200, 250, 300, 350, 400, 450, 500, 550, 600, 700, 800, 900, 1000 millimolar (mM) or more. The eluted polymerase protein can be dialyzed into a buffer containing a neutral pH. The eluted polymerase protein can be dialyzed into a buffer containing a basic pH (e.g. about pH 7.5, pH 8.0, or pH 8.5).

Purification with the biotin-tag (e.g., biotin binding domain tag) can involve coupling of a biotin molecule to the biotin tag in vivo or in vitro by enzymatic biotinylation prior to purification of the polymerase protein through avidin or streptavidin based method. Enzymatic biotinylation may utilize biotin ligase (BirA) to conjugate a biotin molecule to the biotin-tag. The biotin-tag (e.g., biotin binding domain tag) may be a polypeptide tag comprising the amino acid sequence selected from MASSLRQILDSQKIEWRSNAGGAS (SEQ ID NO: 3) or GLNDIFEAQKIEWHE (SEQ ID NO: 5). The biotin-tag MASSLRQILDSQKIEWRSNAGGAS (SEQ ID NO: 3) has the DNA sequence of ATGGCTAGTAGCCTGCGCCAGATCCTGGACAGCCAGAAAATCGAATGGCGCAGCAA CGCTGGTGGTGCTAGT (SEQ ID NO: 4). The biotin-tag (e.g., biotin binding domain tag) may be a polypeptide tag comprising the amino acid sequence MASSLRQILDSQKIEWRSNAGGAS (SEQ ID NO: 3). The biotin-tag (e.g., biotin binding domain tag) may be a polypeptide tag comprising the amino acid sequence GLNDIFEAQKIEWHE (SEQ ID NO: 5). The biotin-tag may be a polypeptide tag consisting of the amino acid sequence MASSLRQILDSQKIEWRSNAGGAS (SEQ ID NO: 3). The biotin-tag may be a polypeptide tag consisting of the amino acid sequence GLNDIFEAQKIEWHE (SEQ ID NO: 5). Biotinylation can be achieved in vivo or in vitro. Sometimes, biotinylation can be achieved in vivo. Sometimes, in vivo biotinylation of a Taq polymerase described herein comprises a biotin-tag of MASSLRQILDSQKIEWRSNAGGAS (SEQ ID NO: 3).

Sometimes, biotinylation can be achieved in vitro. During the biotinylation process, biotin first forms biotinoyl-5′-AMP in the presence of ATP, and biotinoyl-5′-AMP interacts with the epsilon-amine of a lysine residue within the biotin-tag to form an amide bond and this process can be facilitated by the BirA enzyme. Purification of the polymerase protein with avidin or streptavidin resin can be achieved through batch mode or may be performed on a liquid chromatography system (e.g., HPLC or FPLC) using a column immobilized with avidin or streptavidin ligands. Elution of the polymerase protein can be achieved by a change in the environment that is at least immediately adjacent to the avidin/streptaviding-biotin pair. As described supra, the environmental change can be altering the temperature, hydrophobicity, ionic strength, conductivity or pH of the environment. In some cases, elution from avidini/streptaviding resin is achieved with biotin analogs such as desthiobiotin, which competes binding of avidin or streptavidin with biotin. Sometimes, elution from avidin/streptaviding resin is achieved with an elution buffer containing a denaturing agent such as urea or guanidinium chloride, a high ionic strength buffer such as 1M NaCl, or 1M (NH₄)₂SO₄, or an elution buffer that has a pH range from about 2-about 3.

Purification with the Z domain protein (or Z domain moiety) (e.g., Z domain wild type, Zacid₂, or Zbasic₂) can comprise contacting a Taq polymerase solution comprising the Z domain with an IgG immobilized resin in either batch mode or through a column chromatography method. Sometimes, a modified Taq polymerase comprising the Z domain (e.g., Z domain-HIS-Taq) described herein can be purified using a cation exchange chromatography method. The Z domain is an engineered analogue of the IgG-binding domain B of Staphylococcal protein A (SpA) (FIG. 10).

The purified polymerase may be further subjected to an additional step to remove nucleic acid contaminants from the polymerase protein sample.

Removal of Nucleic Acid Contaminants from Biological Samples

Nucleic acid contaminants from PCR reagents (e.g., from polymerases or buffers) can result in false positives in PCR-based methods. One of the contaminating sources can be from polymerases, such as Taq polymerase. As a result, trace amounts of contaminating DNA from a polymerase source (e.g., from the Taq polymerase source) can be co-amplified, leading to misleading, ambiguous, and unreliable results. Further, a high false positive rate can lead to interpretation of the results difficult.

In some aspects, disclosed herein are methods for removal of nucleic acid contaminants from a biological sample. The methods for removal of nucleic acid contaminants from a biological sample may include a protamine-based method, a silica-based method, or a combination thereof. The biological sample may be a cell lysis sample or a culture media sample. The biological sample may be a culture media sample. The culture media sample may comprise secreted protein. The biological sample may be a protein sample. The biological sample may be a polymerase sample. The polymerase may be a DNA polymerase or an RNA polymerase. As described elsewhere herein, exemplary DNA polymerase may include the polymerase from Thermus aquaticus (Taq polymerase), Terminal deoxynucleotidyl transferase transcriptase (RT), or any other polynucleotide polymerase known in the art. Additional polymerases include, but are not limited to, Bst DNA polymerase, Bsu DNA polymerase, Crimson Taq DAN polymerase, Deep Vent_{R}™ DNA polymerase, Deep Vent_{R}™ (exo-) DNA polymerase, E. coli DNA polymerase I, Klenow fragment (3′-5′ exo-), DNA polymerase I (large Klenow fragment), LongAmp® Taq DNA polymerase, LongAmp® Hot Start Taq DNA polymerase, M-MuLV reverse transcriptase; One Taq® DNA polymerase, One Taq® Hot Start DNA polymerase, phi29 DNA polymerase, Phusion® Hot Start Flex DNA polymerase, Phusion® High-Fidelity DNA polymerase, Q5®+Q5® Hot Start DNA polymerase, Sulfolobus DNA polymerase IV, T4 DNA polymerase, Theminator™ DNA polymerase, Vent_{R}™ DNA polymerase, and Vent_{R}™ (exo-) DNA polymerase.

The biological sample may be a Taq polymerase. The Taq polymerase may be a native Taq polymerase or a modified Taq polymerase. The Taq polymerase may contain one or more of the ABS, HIS(6), and Biotin tags described herein. The Taq polymerase may be the Taq polymerase of SEQ ID NO: 1. The Taq polymerase may be cloned and expressed from the Taq polymerase construct described supra.

### Protamine-Based Method

In some instances, disclosed herein is a method for removal of nucleic acid contaminants from a biological sample that comprises contacting a biological sample with protamine-coated beads; and harvesting the biological sample from protamine-coated beads through a separation method to remove the nucleic acid contaminant from the biological sample. Protamine may be covalently or non-covalently bound to the beads. Protamine may be covalently bound to the beads.

Protamine is a small positively charged arginine-rich protein (pI>11) that can interact or bind to nucleic acid. Protamine may be obtained from mammalian, amphibian, or plant sources. Mammalian sources may include human and non-human primates, mice, rat, bull, boar, and the like. Amphibian source may include salmon, herring, rainbow trout, tuna fish, starry sturgeon, and dogfish. Plant sources may include algae, bryophytes, and ferns. Protamine obtained from human may comprise two variants, obtained from protamine encoding genes PRM1 and PRM2. Protamine from fish, such as for example from salmon, may comprise one to about 15 variants encoded by one to about 15 protamine genes. Sometimes, protamine may be obtained from fish. Sometimes, protamine may be obtained from salmon.

Protamine as used herein may comprise native protamine obtained from mammalian, amphibian, or plant sources, or modified protamine such as protamine peptides or protamine fragments. Sometimes, protamine obtained from fish may be referred to as: salmine (salmon), clupeine (herring sperm), iridine (rainbow trout), thinnine (tuna fish), stelline (starry sturgeon), or scylliorhinine (dogfish). Protamine may be obtained from salmon.

Protamine may refer to a homogenous sample of protamine. In some cases, protamine may refer to a heterogeneous sample of protamine. Sometimes, the heterogeneous sample of protamine may comprise multiple protein variants or homologs from a single source, such as a single mammalian, fish, or plant source. Other times, the heterogeneous sample of protamine may comprise multiple protein variants or homologs from two or more sources. Sometimes, the heterogeneous sample of protamine may have an average molecular weight range of from about 3500 to about 10,000 Dalton.

Protamine may be a recombinant protamine. Recombinant protamine may be a mammalian recombinant protamine, recombinant protamine obtained from an amphibian source, or plant recombinant protamine. Sometimes, recombinant protamine may be obtained from fish. Sometimes, recombinant protamine may be obtained from salmon (e.g., Oncorhynchus keta).

Protamine may be a protamine salt, such as for example, protamine sulfate. Protamine sulfate may be obtained from mammalian, amphibian, or plant source, or may be obtained from recombinant protamine. In some instances, protamine sulfate obtained from salmon is referred to as salmine sulfate.

Protamine-coated beads may include agarose beads, acrylamide beads, dextrose beads, magnetic beads, or combinations thereof. The beads may be agarose beads. Exemplary agarose beads may include Sepharose® beads and magnetic Sepharose® bead. The agarose beads (e.g., Sepharose® beads) may include agarose bead conjugates (e.g., Sepharose® bead conjugates). The agarose bead conjugates (e.g., Sepharose® bead conjugates) may contain a functional group for coupling of protamine to the beads. Functional groups may include, such as for example, a N-hydroxysuccinimidyl (NHS)-activating group for coupling to primary amines, an aldehyde functional group for coupling to primary amines, reduced cysteine groups for forming thioether bonds, or carboxyl functional group for coupling to primary or terminal carboxylates (e.g., glutamic acid and aspartic acid). The agarose bead conjugates (e.g., Sepharose® bead conjugates) may include affinity moieties such as for example, Ni²⁺, Co²⁺, biotin, streptavidin, anti-protamine antibody, glutathione-S-transferase (GST), maltose binding protein (MBP), STREP-tag, and the like.

The beads may be acrylamide beads. The acrylamide beads may be magnetic acrylamide beads. The acrylamide beads may include acrylamide bead conjugates. The acrylamide bead conjugates may contain a functional group for coupling of protamine to the beads. Functional groups may include, such as for example, a N-hydroxysuccinimidyl (NHS)-activating group for coupling to primary amines, an aldehyde functional group for coupling to primary amines, reduced cysteine groups for forming thioether bonds, or carboxyl functional group for coupling to primary or terminal carboxylates (e.g., glutamic acid and aspartic acid). The acrylamide bead conjugates may include affinity moieties such as for example, Ni²⁺, Co²⁺, biotin, streptavidin, anti-protamine antibody, glutathione-S-transferase (GST), maltose binding protein (MBP), STREP-tag, and the like.

The beads may be dextrose beads. Exemplary dextrose beads include Sephadex®. The dextrose beads may be magnetic dextrose beads. The dextrose beads may include dextrose bead conjugates. The dextrose bead conjugates may contain a functional group for coupling of protamine to the beads. Functional groups may include, such as for example, a N-hydroxysuccinimidyl (NHS)-activating group for coupling to primary amines, an aldehyde functional group for coupling to primary amines, reduced cysteine groups for forming thioether bonds, or carboxyl functional group for coupling to primary or terminal carboxylates (e.g., glutamic acid and aspartic acid). The dextrose bead conjugates may include affinity moieties such as for example, Ni²⁺, Co²⁺iotin, streptavidin, anti-protamine antibody, glutathione-S-transferase (GST), maltose binding protein (MBP), STREP-tag, and the like.

Protamine may be coupled to the beads through one or more of the above described functional groups. Protamine may be coupled to the beads through NHS-activating group, aldehyde functional group, cysteine group, or carboxyl functional group. Protamine may be coupled to the beads through affinity moieties such as for example, Ni²⁺, Co²⁺iotin, streptavidin, anti-protamine antibody, glutathione-S-transferase (GST), maltose binding protein (MBP), STREP-tag, and the like. Sometimes, protamine may be coupled to the beads through NHS-activating group.

In some instances, a nucleic acid contaminant is removed from a biological sample through incubation of protamine-coated beads with the biological sample and removal of protamine-coated beads from the treated biological sample with a separation method, thereby removing the nucleic acid contaminant. The biological sample may be a protein sample. The biological sample may be a polymerase sample. The biological sample may be a Taq polymerase sample. The biological sample may be a Taq polymerase sample in which the Taq contains a Taq construct described herein.

The incubation time can be from about 2 minutes to about 24 hours. The incubation time can be from about 5 minutes to about 12 hours, or about 5 minutes to about 6 hours. The incubation time can be at least 2, 3, 4, 5, 6, 7, 8, 9, 10, 11, 12, 13, 14, 15, 16, 17, 18, 19, 20, 22, 24, 25, 26, 28, 30, 35, 40, 45, 50, 55, 60, 90, 120, 180 minutes, 4 hours, 5 hours, 6 hours, or more. The incubation time can be at most 2, 3, 4, 5, 6, 7, 8, 9, 10, 11, 12, 13, 14, 15, 16, 17, 18, 19, 20, 22, 24, 25, 26, 28, 30, 35, 40, 45, 50, 55, 60, 90, 120, 180 minutes, 4 hours, 5 hours, 6 hours, or less. The incubation time can be about 5 minutes. The incubation time can be about 10 minutes. The incubation time can be about 15 minutes. The incubation time can be about 20 minutes.

The incubation temperature can be from about 0° C. to about 40° C., from about 4° C. to about 37° C., or about 10° C. to about 25° C. The incubation temperature can be at least 2° C., 4° C., 8° C., 10° C., 15° C., 20° C., 25° C., 30° C., 35° C., 37° C., or more. The incubation temperature can be at most 2° C., 4° C., 8° C., 10° C., 15° C., 20° C., 25° C., 30° C., 35° C., 37° C., or less.

The buffer pH during the incubation time can be from about pH 3 to about pH 10. The buffer pH during the incubation time can be from about pH 4 to about pH 9, from about pH 5 to about pH 8, or from about pH 5 to about pH 7. The buffer pH during the incubation time can be at least 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 7.6, 7.7, 7.8, 7.9, 8, 8.5, 9, 9.5, or more. The buffer pH during the incubation time can be at most 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 7.6, 7.7, 7.8, 7.9, 8, 8.5, 9, 9.5, or less. The buffer pH during the incubation time can be 4, 4.1, 4.2, 4.3, 4.4, 4.5, 4.6, 4.7, 4.8, 4.9, 5, 5.5, 6, 6.5, 7, 7.5, 7.6, 7.7, 7.8, 7.9, or 8. The buffer pH during the incubation time can be about 4, 4.1, 4.2, 4.3, 4.4, 4.5, 4.6, 4.7, 4.8, 4.9, or 5. The buffer pH during the incubation time can be about 4, 4.1, 4.2, 4.3, 4.4, 4.5, or 4.6. The buffer pH during the incubation time can be about 4.4.

The salt concentration of the buffer during the incubation time can be from about 0M to about 2M. Sometimes, the salt concentration can be at least 0.005, 0.01, 0.05, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.5, 0.6, 0.7, 0.8, 0.9, 1, 1.1, 1.2, 1.3, 1.4, 1.5, 1.6, 1.7, 1.8, 1.9, or more. Sometimes, the salt concentration can be at most 0.005, 0.01, 0.05, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.5, 0.6, 0.7, 0.8, 0.9, 1, 1.1, 1.2, 1.3, 1.4, 1.5, 1.6, 1.7, 1.8, 1.9, or less. The salt can be any monovalent and divalent salts suitable for use in a biological buffer and can include, but not limited to, NaCl, KCl, NH₄Cl (NH₄)₂SO₄, K₂SO₄, and the like.

The separation method may include a centrifugation method or a filtration method. Sometimes, the separation method may be a centrifugation method. The centrifugation speed can be from about 500 to about 8000 g, about 600 to about 5000 g, or about 800 to about 3000 g. The centrifugation speed can be at least 500, 600, 700, 800, 900, 1000, 1200, 1400, 1600, 1800, 2000, 2500, 3000, or more. The centrifugation speed can be at most 500, 600, 700, 800, 900, 1000, 1200, 1400, 1600, 1800, 2000, 2500, 3000, or less. The centrifugation speed can be from about 2000 rpm to about 9000 rpm, about 3000 rpm to about 8000 rpm, or about 4000 rpm to about 7000 rpm. The centrifugation speed can be at most 2000, 3000, 4000, 5000, 6000, 7000, 8000 rpm, or more. The centrifugation speed can be at least 2000, 3000, 4000, 5000, 6000, 7000, 8000 rpm, or less.

In some cases, a nucleic acid contaminant is removed from a biological sample through loading the biological sample (e.g., Taq polymerase) onto a protamine charged column and collecting the elution fraction from the protamine charged column, in which the elution fraction is the fraction of the biological sample (e.g., Taq polymerase) without the nucleic acid contaminant. In some instances, the protamine-charged column is further washed with one, two, three, or more column volumes of the loading buffer to further recover the biological sample (e.g., Taq polymerase). In some instances, the nucleic acid contaminant is bound to protamine and is thereby removed from the biological sample (e.g., Taq polymerase).

The treated biological sample (e.g., Taq polymerase) can be about 95%, 96%, 97%, 98%, 99%, 99.5%, 99.9%, 99.99% pure. The treated biological sample (e.g., Taq polymerase) can be at least 95%, 96%, 97%, 98%, 99%, 99.5%, 99.9%, 99.99%, or more pure. The treated biological sample (e.g., Taq polymerase) can be at most 95%, 96%, 97%, 98%, 99%, 99.5%, 99.9%, or 99.99% pure.

Sometimes, the treated biological sample is a polymerase. In some cases, the polymerase (e.g., Taq polymerase) treated by the protamine-based method, does not yield nonspecific amplification. In some instances, the nonspecific amplification is not observed after 10, 15, 20, 25, 30, 35, 40, 45, 50, 55, 60, 65, 70, 80, 90, 100 cycles or more, in a reaction using the treated polymerase. In some cases, the nonspecific amplification is not observed after 10, 15, 20, 25, 30, 35, 40, 45, 50, 55, 60, 65, 70, 80, 90, 100 cycles or more, in a reaction using a treated Taq polymerase described herein.

The treated biological sample can comprise less than about 50 ng/mL, 45 ng/mL, 40 ng/mL, 35 ng/mL, 30 ng/mL, 29 ng/mL, 28 ng/mL, 27 ng/mL, 26 ng/mL, 25 ng/mL, 24 ng/mL, 23 ng/mL, 22 ng/mL, 21 ng/mL, 20 ng/mL, 15 ng/mL, 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or less nucleic acids. The treated biological sample can be a polymerase. Treated polymerase (e.g., Taq polymerase) can comprise less than about 50 ng/mL, 45 ng/mL, 40 ng/mL, 35 ng/mL, 30 ng/mL, 29 ng/mL, 28 ng/mL, 27 ng/mL, 26 ng/mL, 25 ng/mL, 24 ng/mL, 23 ng/mL, 22 ng/mL, 21 ng/mL, 20 ng/mL, 15 ng/mL, 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or less nucleic acids. The Taq polymerase can comprise less than about 50 ng/mL, 45 ng/mL, 40 ng/mL, 35 ng/mL, 30 ng/mL, 29 ng/mL, 28 ng/mL, 27 ng/mL, 26 ng/mL, 25 ng/mL, 24 ng/mL, 23 ng/mL, 22 ng/mL, 21 ng/mL, 20 ng/mL, 15 ng/mL, 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or less nucleic acids. The nucleic acid is DNA or RNA. The nucleic acid is DNA.

The biological sample treated by the protamine-coated beads can be further treated by an electrophoretic method to remove nucleic acid contaminant. The biological sample can be placed in a dialysis bag (e.g., MWCO 1000 D, 2000 D, 3000 D, 5000 D, or 10 kD) and a voltage (e.g., 30V, 35V, 40V, 45V, 50V, 55V, 60V) can be applied for about 5 minutes to about 1 hour, about 8 minutes to about 30 minutes, or about 10 minutes to about 25 minutes.

The biological sample (e.g., Taq polymerase) further treated by the electrophoretic method can be about 95%, 96%, 97%, 98%, 99%, 99.5%, 99.9%, 99.99% pure. The biological sample (e.g., Taq polymerase) further treated by the electrophoretic method can be at least 95%, 96%, 97%, 98%, 99%, 99.5%, 99.9%, 99.99%, or more pure. The biological sample (e.g., Taq polymerase) further treated by the electrophoretic method can be at most 95%, 96%, 97%, 98%, 99%, 99.5%, 99.9%, or 99.99% pure.

The biological sample further treated by the electrophoretic method can be a polymerase. In some cases, the polymerase (e.g., Taq polymerase) further treated by the electrophoretic method, does not yield nonspecific amplification. In some instances, the nonspecific amplification is not observed after 10, 15, 20, 25, 30, 35, 40, 45, 50, 55, 60, 65, 70, 80, 90, 100 cycles or more, in a reaction using the treated polymerase. In some cases, the nonspecific amplification is not observed after 10, 15, 20, 25, 30, 35, 40, 45, 50, 55, 60, 65, 70, 80, 90, 100 cycles or more, in a reaction using a treated Taq polymerase described herein.

The treated biological sample further treated by the electrophoretic method can comprise less than about 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or less nucleic acids. The treated biological sample can be a polymerase. Treated polymerase (e.g., Taq polymerase) further treated by the electrophoretic method can comprise less than about 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or less nucleic acids. The Taq polymerase further treated by the electrophoretic method can comprise less than about 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or less nucleic acids. The nucleic acid is DNA or RNA. The nucleic acid is DNA.

In some instances, the protamine-based method can be used in combination with a silica-based method for removal of nucleic acid contaminant from a biological sample. In some instances, the silica-based method is used prior to using the protamine-based method. Sometimes, the treated biological sample is substantially pure biological sample. In some cases, the treated biological sample is substantially free of nucleic acid contaminant.

Nucleic acid contaminant may be DNA contaminant (e.g., genomic and cDNA), RNA contaminant, or DNA/RNA hybrid contaminant. Nucleic acid or nucleic acid molecule can contain combinations of deoxyribo- and ribo-nucleotides, and combinations of bases including uracil, adenine, thymine, cytosine, guanine, inosine, xanthine hypoxanthine, isocytosine and isoguanine Nucleic acid contaminant may be DNA contaminant. Nucleic acid contaminant may be RNA contaminant.

### Silica-Based Method

Reversible interactions between DNA and silica may be utilized for extraction of DNA from biological samples. Sometimes, DNA-silica interaction can be electrostatically unfavorable, since DNA and silica surface can be both negatively charged under certain experimental conditions. Lowering the solution pH can decrease the silica's negative surface charge density and can thereby reduce the electrostatic repulsion between DNA and silica. To drive adsorption to silica under normal pH conditions, buffers comprising a chaotropic agent or amino acid buffers can be used.

Chaotropic agent is a molecule that can disrupt the hydrogen bonding network between water molecules in a solution. Chaotropic agent can be an organic solvent or a salt. Exemplary chaotropic agent can include guanidinium chloride, guanidinium thiocyante, lithium perchlorate, lithium acetate, magnesium chloride, butanol, ethanol, phenol, propanol, sodium dodecyl sulfate (SDS), thiourea, urea, and the like. Sometimes, the chaotropic agent is guanidinium thiocyante.

An amino acid buffer can comprise one or more amino acids selected from lysine, arginine, histidine, asparagine, glutamine, and glycine. An amino acid buffer can comprise positively charged amino acids. Positively charged amino acids include lysine, arginine, histidine, asparagine and glutamine. An amino acid buffer can comprise lysine, arginine, histidine, asparagine, glutamine, or a combination thereof. An amino acid buffer can comprise arginine, glutamine, glycine, or a combination thereof.

In some instances, a biological sample is incubated in the presence of silica to remove nucleic acid contaminant from the biological sample. Sometimes, a chaotropic agent and/or an amino acid buffer is also added during the incubation step. In some cases, the silica and biological sample solution is further mixed at a temperature of between about 2° C. to about 40° C. for a period of time (e.g., 30 minutes, 1 hour, 2 hours, 3 hours, or more). In some instances, the silica containing the nucleic acid contaminant is removed from the biological sample by a centrifugation method or a filtration method.

Sometimes, the silica purification step can remove up to 50%, 60%, 70%, 80%, 90%, 95%, 99%, or 100% of nucleic acid contaminants in the biological sample (e.g., culture media). The silica purification step can remove up to 80%, 90%, 95%, 99%, or 100% of nucleic acid contaminants in the biological sample (e.g., culture media). In some instances, the silica purification step processes the biological sample prior to the protamine-based method.

As described elsewhere herein, the biological sample may be a cell lysis sample or a culture media (or growth media) sample. The biological sample may be a culture media (or growth media) sample. The culture media sample may comprise secreted protein. The biological sample may be a protein sample. The biological sample may be a polymerase sample. The polymerase may be a DNA polymerase or an RNA polymerase. The biological sample may be a Taq polymerase. The Taq polymerase may be a native Taq polymerase or a modified Taq polymerase. The Taq polymerase may contain one or more of the ABS, HIS, Z domain, and/or Biotin tags described herein. The Taq polymerase may be cloned and expressed from the Taq polymerase construct described supra.

Amplification Reaction with Polymerase and an Albumin

During an amplification reaction, a polymerase can initiate non-specific amplification at a temperature below a polymerase extension temperature. Sometimes, a polymerase inhibitor is added to the amplification reaction mixture to inhibit the activity of the polymerase below polymerase extension temperature. At a temperature at or above polymerase extension temperature, the polymerase inhibitor is inactivated and the polymerase activity is restored. Described herein is a reaction mixture that comprises (a) a polymerase comprising an albumin binding moiety; and (b) an albumin. In some aspects, the inhibition of a polymerase (e.g., Bio-HIS-ABS-Taq or Bio-HIS-Z domain-Taq described herein) by the addition of albumin (e.g., human serum albumin) can be referred to as a Hot Start process. The combination of a polymerase (e.g., Bio-HIS-ABS-Taq or Bio-HIS-Z domain-Taq described herein) and albumin (e.g., human serum albumin) can be referred to as a Hot Start mixture. The polymerase (e.g., Bio-HIS-ABS-Taq or Bio-HIS-Z domain-Taq described herein) can be referred to as a Hot Start polymerase.

The albumin can inhibit the activity of the polymerase by binding to the polymerase at a temperature of from about 0° C. to about 60° C., from about 20° C. to about 55° C., or from about 25° C. to about 50° C. The albumin can inhibit the activity of the polymerase by binding to the polymerase at a temperature of at most 60, 59, 58, 57, 56, 55, 54, 53, 52, 51, 50, 49, 48, 47, 46, 45, 44, 43, 42, 41, 40, 35, 30, 25, 20, 15, 10, or 5° C. The temperature at which the albumin can inhibit the activity of the polymerase can be at about 60, 59, 58, 57, 56, 55, 54, 53, 52, 51, 50, 49, 48, 47, 46, 45, 44, 43, 42, 41, 40, 35, 30, 25, 20, 15, 10, or 5° C. Albumin can inhibit the activity of the polymerase during an annealing step of an amplification reaction. Albumin can inhibit the activity of the polymerase during a reaction setup.

The albumin can be released from the polymerase at a temperature of at least 61° C. or higher. The albumin can be released from the polymerase at a temperature of at least 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 95, 100, 105, 110, 115, 120, 125, 130 ° C. or higher. Albumin can be released from the polymerase at an extension step of an amplification step.

The polymerase can regain its enzymatic activity at a temperature of at least 61° C. or higher. The polymerase can regain its enzymatic activity at a temperature of at least 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 95, 100, 105, 110, 115, 120, 125, 130 ° C. or higher.

The albumin can inhibit the activity of the polymerase by about 10% to about 100% relative to a control. The albumin can inhibit the activity of the polymerase by at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90%, or more relative to a control. For example, the control can be the activity of an equivalent polymerase in the absence of a polymerase inhibitor and can be set at 100%. The albumin can inhibit the activity of the polymerase by about 10%, 11%, 12%, 20%, and so forth. Sometimes, the control can be the activity of an equivalent polymerase without the presence of a polymerase inhibitor, such as albumin. The temperature at which the albumin can inhibit the activity of the polymerase can be at about 60, 59, 58, 57, 56, 55, 54, 53, 52, 51, 50, 49, 48, 47, 46, 45, 44, 43, 42, 41, 40, 35, 30, 25, 20, 15, 10, or 5° C.

The activity of the polymerase in the presence of an albumin can be a reduced activity. The reduced activity can be measured as a percentage relative to a control. The control can be the activity of an equivalent polymerase in the absence of a polymerase inhibitor. The reduced activity of the polymerase can be at most about 5, 10, 11, 12, 13, 14, 15, 16, 17, 18, 19, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 95%, or less relative to a control. For example, the control can be the activity of an equivalent polymerase in the absence of a polymerase inhibitor and can be set at 100%. The polymerase can have a reduced activity of at most about 5, 10, 11, 12, 13, 14, 15, and so forth compared to the 100% activity of the control. Sometimes, the control can be the activity of an equivalent polymerase without the presence of a polymerase inhibitor, such as albumin. The temperature at which the albumin can inhibit the activity of the polymerase can be at about 60, 59, 58, 57, 56, 55, 54, 53, 52, 51, 50, 49, 48, 47, 46, 45, 44, 43, 42, 41, 40, 35, 30, 25, 20, 15, 10, or 5° C.

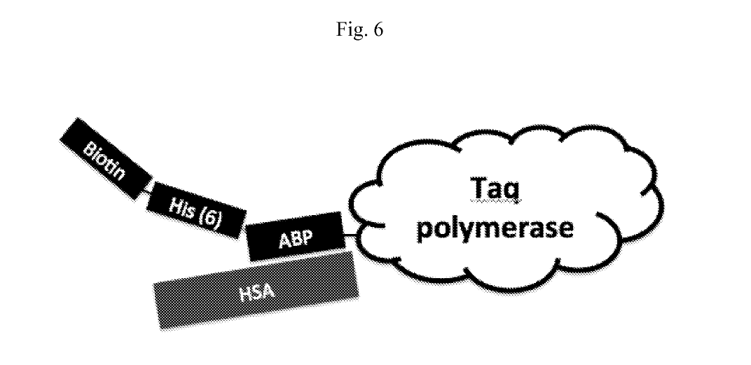

As described elsewhere herein, the polymerase can be a DNA polymerase or an RNA polymerase. The polymerase can comprise Taq polymerase, Vent polymerase, Klenow Fragment (3′-5′ exo-), DNA Polymerase I (large Klenow fragment), E. coli DNA polymerase I, phi29 DNA polymerase, Phusion DNA polymerase, or T4 DNA polymerase. The polymerase can be a Taq polymerase. Described herein is a Taq polymerase comprising an albumin binding moiety and an albumin (FIG. 6). Taq polymerase can be native or modified Taq polymerase.

The albumin binding moiety can be directly connected to the Taq polymerase or can be connected to the Taq polymerase though a spacer. A genetic sequence of the albumin binding moiety and the Taq polymerase can comprise the albumin binding moiety sequence residing on the 3′ end of the Taq polymerase sequence, residing on the 5′ end of the Taq polymerase sequence, or residing on both the 3′ end and 5′ end of the Taq polymerase sequence. The albumin can be mammalian albumin or a mammalian albumin analogue. The albumin can be human serum albumin. The albumin can be bovine serum albumin.

The albumin binding moiety able to bind serum albumin can be at least a part of Streptococcal protein G. The at least a part of Streptococcal protein G can be the entire Streptococcal protein G. The at least a part of Streptococcal protein G can comprise ABP (112aa), BB (214aa), ABD (46aa), ADB1 binding site, ADB2 binding site, or ADB3 binding site. The ABD to albumin affinity can be 1.5 nanomolar or less. The ABD to human serum albumin affinity can be 1.5 nanomolar or less.

The polymerase can further comprise a HIS moiety, a biotin-tag moiety, Z domain (or Z domain moiety), or a combination thereof. The polymerase can have the sequence as illustrated in SEQ ID NO: 1.

The reaction mixture can be an amplification reaction mixture. The amplification can be a polymerase chain reaction (PCR). The amplification can comprise whole genome amplification, helicase dependent amplification, nicking enzyme amplification reaction, reverse transcription PCR (RT-PCR), ligation mediated PCR, methylation specific PCR, digital PCR, hot start PCR, multiplex ligation-dependent probe amplification (MLPA), multiplex-PCR, nested PCR, overlap-extension PCR, or quantitative PCR (qPCR). The amplification can be a next-generation sequencing method.

### Characterization of Polymerase Activity

Assays for characterization or determination of polymerase activity can be a radioactivity based assay which uses radiolabeled nucleotides to evaluate polymerase activity, or a fluorescence-based assay which replaces radioactive isotopes with fluorescent dyes to evaluate polymerase activity. Described herein is an assay that utilizes an oligonucleotide of Formula (I) to characterize or determine a DNA polymerase activity (e.g., Taq polymerase activity).

The assay described herein can allow real time monitoring of a DNA polymerase activity (e.g., Taq polymerase activity) without the use of additional fluorescent dyes or radiolabeled reagents. For example, the assay can take advantage of the rate of production of inorganic pyrophosphate (PPi) using an oligonucleotide of Formula (I) as a substrate, utilizing the following reactions:

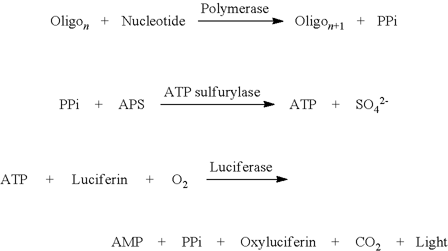

In general, released PPi in the reaction catalyzed by a DNA polymerase (e.g., Taq polymerase) can be subsequently converted to ATP by ATP sulfurylase in the presence of adenosine phosphosulphate (APS). The ATP produced in this step can participate in the reaction involving oxidation of luciferase and generation of light. The light generated during this step can be determined using a luminometer. In some instances, an excess of ATP sulfurylase and luciferase are used to saturate the sulfurylase and luciferase activities. As such, the rate of light production can be solely dependent on the rate of PPi production (i.e. catalytic activity/concentration of DNA polymerase), which can allow characterization and determination of the DNA polymerase activity based on a plot of luminescence increase based on time (see Example 4 and FIG. 8).

The oligonucleotide of Formula (I) is illustrated as S-D (Formula I), wherein S can be a single stranded oligonucleotide between about 20 to about 300 nucleotides in length with a non-self-complementary sequence, D can be an oligonucleotide consisting of a self-complementary sequence that forms a double-stranded oligonucleotide, and S can be covalently attached to D. S can be a single stranded oligonucleotide between about 20 to about 200, about 20 to about 100, about 20 to about 80, about 20 to about 50, or about 20 to about 30 nucleotides in length with a non-self-complementary sequence. S can be a single stranded oligonucleotide between about 20 to about 200 nucleotides in length with a non-self-complementary sequence. S can be a single stranded oligonucleotide about 20, 21, 22, 23, 24, 25, 26, 27, 28, 29, 30, 40, 50, 60, 70, 80, 90, 100, 150, or more nucleotides in length with a non-self-complementary sequence. D can be an oligonucleotide between about 20 to about 100, about 25 to about 80, about 30 to about 60, or about 40 to about 50 nucleotides in length consisting of a self-complementary sequence that forms a double-stranded oligonucleotide. D can be an oligonucleotide between about 40 to about 50 nucleotides in length consisting of a self-complementary sequence that forms a double-stranded oligonucleotide. D can further contain a hairpin. The hairpin can be about 3, 4, 5, 6, 7, 8, 9, 10 or more nucleotides in length. The hairpin can be about 3 nucleotides in length. S and D can be covalently attached through the phosphate backbones. The oligonucleotide further comprises a biotin.

Sometimes D can be GC rich, leading to increased hydrogen bonding and resulting in a high melting temperature (e.g., 83° C.) to allow the assay to be run at temperatures desirable for DNA polymerase (e.g., Taq polymerase) activity determination. Sometimes, the sequence of S does not interact with the sequence of D. As such, this can lead to low or minimal alternative oligonucleotide conformations during amplification.

The single stranded oligonucleotide described above can have the sequence 5′-TTTTTGCATGGTAATTCGTCAGACTGG-3′ (SEQ ID NO: 6). The sequence of S can be about 70%, 75%, 80%, 85%, 90%, 95%, or 99% identical to SEQ ID NO: 6. The sequence of S can be about 90% identical to SEQ ID NO: 6. The sequence of S can be about 95% identical to SEQ ID NO: 6. The sequence of S can be about 99% identical to SEQ ID NO: 6. The sequence length of S can be about 70%, 75%, 80%, 85%, 90%, 95%, or 99% identical to SEQ ID NO: 6. The sequence length of S can be about 90% identical to SEQ ID NO: 6. The sequence length of S can be about 95% identical to SEQ ID NO: 6. The sequence length of S can be about 99% identical to SEQ ID NO: 6. The sequence of S can be SEQ ID NO: 6.

Sometimes, the sequence of D can be about 70%, 75%, 80%, 85%, 90%, 95%, or 99% identical to sequence: 5′-GCCGTCGCGCTTTTACAACGGAACGTTGTAAAAGCGCGACGGC-3′ (SEQ ID NO: 7). The sequence of D can be about 90% identical to SEQ ID NO: 7. The sequence of D can be about 95% identical to SEQ ID NO: 7. The sequence of D can be about 99% identical to SEQ ID NO: 7. The sequence length of D can be about 70%, 75%, 80%, 85%, 90%, 95%, or 99% identical to SEQ ID NO: 7. The sequence length of D can be about 90% identical to SEQ ID NO: 7. The sequence length of D can be about 95% identical to SEQ ID NO: 7. The sequence length of D can be about 99% identical to SEQ ID NO: 7. The sequence of D can be SEQ ID NO: 7.

In some instances, the oligonucleotide of Formula (I) is selected from 5′-TTT TTG CAT GGT AAT TCG TCA GAC TGG GCC GTC GCG CTT TTA CAA CGG AAC GTT GTA AAA GCG CGA CGG C-3′ (SEQ ID NO: 8), 5′-Biotin-TTT TTG CTG GAA TTC GTC AGA CTG GCC GTC GTT TTA CAA CGG AAC GTT GTA AAA CGA CGG-3′ (SEQ ID NO: 9), or 5′-Biotin-TTT TTC CCC CTT TTT GGG GGA AAA ACC GTC GTT TTA CAA CGG AAC GTT GTA AAA CGA CGG-3′ (SEQ ID NO: 10).

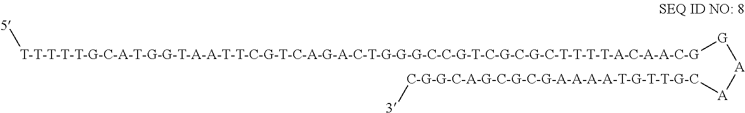

The oligonucleotide of Formula (I) can be SEQ ID NO: 8. The oligonucleotide of Formula (I) can be SEQ ID NO: 9. The oligonucleotide of Formula (I) can be SEQ ID NO: 10.

### Applications in the Field of Molecular Diagnostics

In some aspects of the invention, the invention is useful in molecular diagnostic fields such as infectious agent identification, hereditary diseases, cancer genetic testing, and genetic variations such as single-nucleotide polymorphism. The invention disclosed herein may be useful for identification of infectious agents. As used herein, an infectious agent is an agent such as a virus, a bacterium, a fungus, a nematode or a protozoan that causes an infection in a host organism such as a mammal, such as a human.

Exemplary infectious virus include: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses'); Poxyiridae (variola viruses, vaccinia viruses, pox viruses), Iridoviridae (e.g., African swine fever virus), or unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Exemplary infectious bacteria include Helicobacter pyloris, Borelia burgdorferi, Legionella pneumophilia, Mycobacteria spp. (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidum, Treponema pertenue, Leptospira, or Actinomyces israelli.

Exemplary infectious fungi include: Cryptococcus neoformans, Histoplasma capsulatum, Coccidioides immitis, Blastomyces dermatitidis, Chlamydia trachomatis or Candida albicans.

Exemplary infectious nematode include: ascarids (Ascaris), filarias, hookworms, pinworms, roundworms or whipworms.

Exemplary infectious protozoan include Acanthamoeba, Balamuthia mandrillaris, Endolimax, Entamoeba histolytica, Giardia lamblia or Plasmodium spp.

In other aspects, invention disclosed herein may be useful for real time monitoring of amplification process, with the use of biotin-conjugated chromophore molecules. For example, instead of chromophore labeled probes (e.g., Molecular Beacons or TaqMan® probes), one or more of a Taq polymerase described herein can be labeled with chromophore-conjugated biotin which can subsequently be used to monitor an amplification process.

### Compositions and Kits

This disclosure also provides compositions and kits for use with the methods described herein. The compositions may comprise any component, reaction mixture and/or intermediate described herein, as well as any combination thereof. For example, the disclosure provides detection reagents for use with the methods provided herein. Any suitable reagent may be provided, including ABS polymerase (e.g., DNA polymerase such as Taq polymerase), HIS(6) and/or ABS polymerase (e.g., DNA polymerase such as Taq polymerase), or HIS(6), ABS, and/or biotin-tag polymerase (e.g., DNA polymerase such as Taq polymerase). Additional detection reagents may include primers for hybridization to target DNA, deoxynucleotides, deoxynucleotide analogues, test target DNA such as oligonucleotides described by oligonucleotide of Formula (I) or test primers complimentary to the test target DNA.

The disclosure may further provide regents for use with the method of nucleic acid contaminant removal described herein. As such, any suitable reagents may be provided, including protamine-coated beads, dialysis bags, or silica resin. Additional suitable reagents may include polymerase such as DNA polymerase. Additional suitable reagents may include one or more Taq polymerases described herein, such as ABS polymerase, HIS(6) and/or ABS polymerase, or HIS(6), ABS, and/or biotin-tag polymerase.

### Instrumentations

In some aspects of the invention, the invention comprises a genetic detection machine, device, apparatus or system. Such machine, device, apparatus or system may comprise one or more of the following: compartments for the abovementioned specialized reagents, sample preparation compartment, reservoirs, mechanism for the detection of bioluminescence (e.g. optical detector), a heating element, a cooling element, a sample moving element (i.e. pushing or suction devise such as one or more pumps), a sample mixing element (i.e. stirrer, mixer, vortex), channels (e.g. closed channels, open channels, microfluidic channels), a specialized computer incorporating bioinformatics software, software and an output device (e.g. sound, display, vibration or printer). In some examples, the software allows controls of the operation of programming and running methods of one or the more compartments or instruments, monitoring the status or processing of the results. Sometimes, the software further allows communication with one or more additional machines, devices, apparatus or systems or a centralized command system, or any combination thereof.

Sometimes, the reagents comprise a DNA polymerase (e.g., Taq polymerase) described herein, nucleosides, target polynucleotide to be amplified and sequenced, and polynucleotide primers. Sometimes, the methods are modified that the amplified DNA becomes immobilized or is provided with means for attachment to a solid support. For example, a PCR primer may be immobilized or be provided with means for attachment to a solid support. Also, vectors may comprise means for attachment to a solid support.

In some aspects of the invention, disclosed is a system for polynucleotide sequencing comprising amplifying at least one polynucleotide to be amplified by hybridizing nucleoside-polyphosphate molecule to at least one polynucleotide to be amplified in a complementary fashion, and linking the hybridized nucleoside-polyphosphate molecule to form polynucleotide strand complementary to at least one polynucleotide to be amplified. The linkage may be covalent linkage. Sometimes, the amplification process involves the release of a pyrophosphate. The pyrophosphate can be further involved in generation of ATP, which can initiate the oxidation of luciferase and generation of light. The generation of light can be used for evaluation of the activity of the polymerase. The system may contain integrated modules in which each module is tasked with, for example, sample preparation, amplification, or reaction monitoring. One or more of the modules may contain specialized software which allow for completion of the tasks for the one or more modules. The system may contain separate units, which functions either individually to complete a portion of the processes disclosed in the invention, or functions in tandem to complete the processes disclosed in the invention. Each individual unit may be tasked with, for example, sample preparation such as a preparation kit, amplification such as a PCR machine, or reaction monitoring such as with a luminometer. One or more of the individual units may contain different software.

The present disclosure provides computer control systems that are programmed to implement methods of the disclosure. FIG. 7 shows a computer system 1001 that is programmed or otherwise configured to control the genetic detection system. The computer system 1001 can regulate various aspects of the flow of the single fluid phase within the genetic detection system of the present disclosure, such as, for example, control various components of the genetic detection system to detect polynucleotide sequence such as single stranded or double stranded polynucleotides (such as RNA, DNA or any modified or non-natural polynucleotide sequence). The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. Examples of operations performed by the CPU 1005 can include fetch, decode, execute, and write back.

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., operator or end user). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030. In some cases, the end user is a lab technician, a physician, a customer, a patient, or a researcher.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display that comprises a user interface (UI) for providing, for example, various aspects of the genetic systems. An example for an aspect can be the level of a polynucleotide detected by the system. The display may optionally include the absolute or relative polynucleotide levels, the sequence of the polynucleotides and data regarding any genetic alterations as well as historical genetic data of the user or any comparative or normal polynucleotide data. Any of the abovementioned polynucleotide levels, as well as the corresponding historical or comparative date and time in which the levels were collected, can be saved in any of the abovementioned storage systems or their combination, and can be accessed by the computer system and/or by a user. Saving may be effectuated by the computer system, by a user, or by both. The historical data may be accessible by the computer system, by a user, or by both. The historical levels displayed may be of a past date and/or time chosen by the user, or of a predetermined date and/or time. The display may also comprise a sketch of all the components of the genetic detection system. The sketch may display the current operational status of the particular component. The sketch may display the level of reactants, polynucleotides, solvents, buffers, enzymes, any other property of the fluid within the system, or any combination thereof. The sketch may also display the abovementioned real time levels, historical levels, or both. The display may further include a user interface that may allow manual control of any of the components of the hypersensitive genetic detection system. Such user control may be effectuated by the user using a touch screen, a remote control device, computer “mouse,” keypad, keyboard, touchpad, stylus, joystick, thumb wheel, voice recognition interface, any other user input interface known in the art, or a combination thereof. Examples of UI's include, without limitation, a graphical user interface (GUI) and web-based user interface.

Methods and systems of the present disclosure can be implemented by way of one or more algorithms. An algorithm can be implemented by way of software upon execution by one or more computer processors. In some examples, one or more algorithms for comparing or evaluating sequencing data may be used.

While some illustrations of the present invention have been shown and described herein, it will be obvious to those skilled in the art that such illustrations are provided by way of example only. It is not intended that the invention be limited by the specific examples provided within the specification. While the invention has been described with reference to the aforementioned specification, the descriptions and illustrations of the examples herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the examples of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

While some illustrations of the present invention have been shown and described herein, it will be obvious to those skilled in the art that such illustrations are provided by way of example only. Numerous variations, changes, and substitutions will now occur to those skilled in the art without departing from the invention. It should be understood that various alternatives to the illustrations of the invention described herein may be employed in practicing the invention. It is intended that the following claims define the scope of the invention and that methods and structures within the scope of these claims and their equivalents be covered thereby.

### EXAMPLES

These examples are provided for illustrative purposes only and not to limit the scope of the claims provided herein.

### Example 1

### Taq Polymerase Purification

E. coli DH5a was used for the E. coli cloning experiments. E. coli Rosetta 2 (DE3) (EMD Millipore, Billerica, Mass., USA) was used as host for the expression experiments. P. pastoris strains KM71, SMD1168 or GS115 were used as host for the yeast expression experiments.

The gene encoding Taq polymerase was amplified from Thermus aquaticus strain YT-1. The gene was cloned into vector pET21, which contains the coding sequence for an N-terminal fusion to the affinity tag Bio-His₆-ABP.

Alternatively, the gene encoding Taq polymerase with an N-terminal fusion to albumin-binding protein (ABP) was synthesized by Genscript (Piscataway, N.J., USA) with the codons optimized for expression in P. pastoris. Flanking XhoI/NotI restriction sites were added such that the coding sequence could be inserted into the vector YpDC541 to create a fusion with the a-mating secretion signal of S. cerevisiae and to be under the control of the methanol-inducible alcohol oxidase promoter. The YpDC541-ABP-Taq construct was integrated into P. pastoris strains KM71, SMD1168 or GS115.

Growth, Expression, and Purification using the E. coli System

E. coli Rosetta 2 (DE3) cells harboring the plasmid pET21-Biotin-His₆-ABP-Taq were used for expression experiments. Cells were grown at 37° C. in Terrific Broth (47.6 g/l, Sigma-Aldrich, St. Louis, Mo., USA) supplemented with 100 μg/ml of carbenicillin and 34 μg/ml of chloramphenicol until OD₆₀₀ reached 0.6. Isopropyl-β-D-thiogalactoside (IPTG) and D-biotin were added at final concentrations of 0.5 mM and 0.1 mM, respectively. Cells were grown an additional 4 hr at 37° C.

Following recombinant expression of Bio-His₆-ABP-Taq in E. coli, the protein was purified by affinity chromatography. The cells were first centrifuged and re-suspended to 1:20 of the original starting volume using wash buffer (50 mM Tris-HCl, pH 8.0, 0.2 M NaCl, 0.05% Tween 20, and 1 mM EDTA). Lysozyme (1 mg/ml), DNAse I (100 U), MgCl₂ (2.5 mM), and CaCl₂ (0.5 mM) were added, and the suspension was incubated at 37° C. for 2 hr. The cells were sonicated, heated to 75° C. for 1 hr, and then centrifuged at 10,000 × g for 25 min. After centrifugation, the supernatant was filtrated (0.22 μm) prior to loading onto a 5 ml human serum albumin (HSA)-Sepharose column, which had been made using HSA (Sigma-Aldrich, St. Louis, Mo., USA) and NHS-Sepharose (GE Healthcare, Pittsburgh, Pa., USA). After loading, the column was washed with 150 ml of washing buffer (50 mM Tris-HCl, pH 8.0, 0.2 M NaCl, 0.05% Tween 20, and 1 mM EDTA) followed by a high salt wash buffer (washing buffer with 2 M NaCl) to remove residual DNA contaminants. A pre-elution wash (10 mM NH₄Ac, pH 5.5) of 50 ml was applied next, followed by elution with 10 ml of 0.5 M HAc, pH 2.8. The eluted sample was collected in 1 ml of 1 M Tris-HCl, pH 8.0 and buffer exchanged by ultrafiltration centrifugal concentrators (Vivaproducts, Littleton, Mass., USA) into 2x storage buffer (40 mM Tris-HCl, pH 8.0, 200 mM KCl, 0.2 mM EDTA, 2 mM dithiothreitol). Glycerol, Tween 20, and IGEPAL CA-360 were added to final concentrations of 50%, 0.5%, and 0.5%, respectively, and the sample was stored at −20° C.

Growth, Expression, and Purification using the Yeast System

Yeast cultures were grown according to the protocols in the Pichia Expression Kit manual (Invitrogen, Carlsbad, Calif., USA). For biomass accumulation, cultures were grown in YPD medium (1% yeast extract, 2% peptone, 2% glucose) at 30° C. until OD₆₀₀ reached >10. For protein expression, the yeast cells were centrifuged, resuspended at OD₆₀₀ of 10 in BMMY medium (1% yeast extract, 2% peptone, 100 mM potassium phosphate pH 6.0, 1.34% YNB, 4×10⁻⁵% biotin, 1% methanol), and were grown at 30° C. for 2-3 days with an addition of 1% methanol supplementation every 24 hr.

Following recombinant expression of ABP-Taq in P. pastoris, the protein was purified by affinity chromatography. The culture was first centrifuged to remove cells and then passed through a 0.22 μm filter. This solution was then applied to a 5 ml human serum albumin (HSA)-Sepharose column, which had been made using HSA (Sigma-Aldrich, St. Louis, Mo., USA) and NHS-Sepharose (GE Healthcare, Pittsburgh, Pa., USA). After loading, the column was washed with 150 ml of washing buffer (50 mM Tris-HCl, pH 8.0, 0.2 M NaCl, 0.05% Tween 20, and 1 mM EDTA) followed by a high salt wash buffer (washing buffer with 2 M NaCl) to remove residual DNA contaminants. A pre-elution wash (10 mM NH₄Ac, pH 5.5) of 50 ml was then applied, followed by elution with 10 ml of 0.5 M HAc, pH 2.8. The eluted sample was collected in 1 ml of 1 M Tris-Hcl, pH 8.0 and buffer exchanged by ultrafiltration centrifugal concentrators (Vivaproducts, Littleton, Mass., USA) into 2× storage buffer (40 mM Tris-HCl, pH 8.0, 200 mM KCl, 0.2 mM EDTA, 2 mM dithiothreitol). Glycerol, Tween 20, and IGEPAL CA-360 were added to final concentrations of 50%, 0.5%, and 0.5%, respectively, and the sample was stored at −20° C.

FIG. 8 illustrates gel electrophoresis of the Taq polymerase-ABS construct following purification from either E. coli or yeast cells. FIG. 8A shows a Coomassie-stained SDS-PAGE gel that shows a Bio-His₆-ABP-Taq polymerase produced in E. coli. Following cell lysis, heating, and centrifugation, the cell lysate was passed over an HSA-Sepharose column for one-step affinity purification. Lane M is the molecular weight ladder (Fisher Scientific, Pittsburgh, Pa., USA). Lane 1 is the cell lysate. Lane 2 is the column flow through. Lane 3 is the column elution. The expected molecular weight of Bio-His₆-ABP-Taq is 113 kDa. FIG. 8B illustrates a gel electrophoresis of amplified DNA. Lines 3 and 4 represent DNA amplified using Taq Polymerase protein construct with ABP and HIS(6), expressed in E. coli, in the absence of anti Taq Polymerase antibodies. FIG. 8C compares the Taq polymerase (Ninja Taq) with several commercially available Taq polymerases. FIG. 8D compares the Taq polymerase (Ninja Taq) with several commercially available Taq polymerases under different concentrations of BSA or HSA. FIG. 8E illustrates a Coomassie-stained SDS-PAGE gel that shows ABP-Taq polymerase produced in P. pastoris. Following centrifugation and filtration, the cell culture supernatant was passed over an HSA-Sepharose column for one-step affinity purification. Lane 1 is column flow-through and lane 2 is column elution. The expected molecular weight of ABP-Taq is 109 kDa. FIG. 8F illustrates an agarose gel showing PCR amplification by Taq polymerase produced in P. pastoris and E. coli. Lanes 1, 2, and 3 illustrate product from batch 1 of Taq polymerase produced in P. pastoris at 1 μl, 0.2 μl, and 0.04 μl of enzyme in 25 μl PCR reactions. Lanes 4, 5, and 6 illustrate product from batch 2 of Taq polymerase produced in P. pastoris at 1 μl, 0.2 μl, and 0.04 μl of enzyme in 25 μl PCR reactions. Lanes 7, 8, and 9 illustrate product from Taq polymerase produced in E. coli at 1 μl, 0.2 μl, and 0.04 μl of enzyme in 25 μl PCR reactions.

Table 1 illustrates the protein and construct Taq polymerase sequences.

TABLE 1

| | |
|---|---|
| Bio-HIS- | MASSLRQILDSQKIEWRSNAGGASHHHHHHGGASLAEAKVLANRELDKYGVSDYHKNLINNA |
| ABS-Taq | KTVEGVKDLQAQVVESAKKARISEATDGLSDFLKSQTPAEDTVKSIELAEAKVLANRELDKY |
| polymerase | GVSDYYKNLINNAKTVEGVKALIDEILAALPGTFAHYMDPNLEALFQGPNSLPLFEPKGRVL |
| (SEQ ID NO: 1) | LVDGHHLAYRTFHALKGLTTSRGEPVQAVYGFAKSLLKALKEDGDAVIVVFDAKAPSFRHEA |
| | YGGYKAGRAPTPEDFPRQLALIKELVDLLGLARLEVPGYEADDVLASLAKKAEKEGYEVRIL |
| | TADKDLYQLLSDRIHVLHPEGYLITPAWLWEKYGLRPDQWADYRALTGDESDNLPGVKGIGE |
| | KTARKLLEEWGSLEALLKNLDRLKPAIREKILAHMDDLKLSWDLAKVRTDLPLEVDFAKRRE |
| | PDRERLRAFLERLEFGSLLHEFGLLESPKALLEAPWPPPEGAFVGFVLSRKEPMWADLLALA |
| | AARGGRVHRAPEPYKALRDLKEARGLLAKDLSVLALREGLGLPPGDDPMLLAYLLDPSNTTP |
| | EGWPGRYGGEWTEEAGERAALSERLFANLWGRLEGEERLLWLYREVERPLSAVLAHMEATGV |
| | RLDVAYLRALSLEVAELIARLEALVFRLAGHPFNLNSRDQLERVLFDELGLPAIGKTEKTGK |
| | RSTSAAVLEALREAHPIVEKILQYRELTKLKSTYIDPLPDLIHPRTGRLHTRFNQTATATGR |
| | LSSSDPNLQNIPVRTPLGQRIRRAFIAEEGWLLVALDYSQIELRVLAHLSGDENLIRVFQEG |
| | RDIHTETASWMFGVPREAVDPLMRRAAKTINFGVLYGMSAHRLSQELAIPYEEAQAFIERYF |
| | QSFPKVRAWIEKTLEEGRRRGYVETLFGRRRYVPDLEARVKSVREAAERMAFNMPVQGTAAD |
| | LMKLAMVKLFPRLEEMGARMLLQVHDELVLEAPKERAEAVARLAKEVMEGVYPLAVPLEVEV |
| | GIGEDWLSAKE |
| Construct | ATGGCTAGTAGCCTGCGCCAGATCCTGGACAGCCAGAAAATCGAATGGCGCAGCAACGCTGG |
| Seq. | TGGTGCTAGTCACCACCACCACCACCACGGTGGTGCTAGCTTAGCTGAAGCTAAAGTCTTAG |
| (SEQ ID NO: 2) | CTAACAGAGAACTTGACAAATATGGAGTAAGTGACTATCACAAGAACCTAATCAACAATGCC |
| | AAAACTGTTGAAGGTGTAAAAGACCTTCAAGCACAAGTTGTTGAATCAGCGAAGAAAGCGCG |
| | TATTTCAGAAGCAACAGATGGCTTATCTGATTTCTTGAAATCACAAACACCTGCTGAAGATA |
| | CTGTTAAATCAATTGAATTAGCTGAAGCTAAAGTCTTAGCTAACAGAGAACTTGACAAATAT |
| | GGAGTAAGTGACTATTACAAGAACCTAATCAACAATGCCAAAACTGTTGAAGGTGTAAAAGC |
| | ACTGATAGATGAAATTTTAGCTGCATTACCTGGTACCTTCGCTCACTACATGGATCCGAATT |
| | TGGAAGCTCTGTTCCAGGGTCCGAATTCGCTGCCCCTCTTTGAGCCCAAGGGCCGGGTCCTC |
| | CTGGTGGACGGCCACCACCTGGCCTACCGCACCTTCCACGCCCTGAAGGGCCTCACCACCAG |
| | CCGGGGGGAGCCGGTGCAGGCGGTCTACGGCTTCGCCAAGAGCCTCCTCAAGGCCCTCAAGG |
| | AGGACGGGGACGCGGTGATCGTGGTCTTTGACGCCAAGGCCCCCTCCTTCCGCCACGAGGCC |
| | TACGGGGGGTACAAGGCGGGCCGGGCCCCCACGCCGGAGGACTTTCCCCGGCAACTCGCCCT |
| | CATCAAGGAGCTGGTGGACCTCCTGGGGCTGGCGCGCCTCGAGGTCCCGGGCTACGAGGCGG |
| | ACGACGTCCTGGCCAGCCTGGCCAAGAAGGCGGAAAAGGAGGGCTACGAGGTCCGCATCCTC |
| | ACCGCCGACAAAGACCTTTACCAGCTCCTTTCCGACCGCATCCACGTCCTCCACCCCGAGGG |
| | GTACCTCATCACCCCGGCCTGGCTTTGGGAAAAGTACGGCCTGAGGCCCGACCAGTGGGCCG |
| | ACTACCGGGCCCTGACCGGGGACGAGTCCGACAACCTTCCCGGGGTCAAGGGCATCGGGGAG |
| | AAGACGGCGAGGAAGCTTCTGGAGGAGTGGGGGAGCCTGGAAGCCCTCCTCAAGAACCTGGA |
| | CCGGCTGAAGCCCGCCATCCGGGAGAAGATCCTGGCCCACATGGACGATCTGAAGCTCTCCT |
| | GGGACCTGGCCAAGGTGCGCACCGACCTGCCCCTGGAGGTGGACTTCGCCAAAAGGCGGGAG |
| | CCCGACCGGGAGAGGCTTAGGGCCTTTCTGGAGAGGCTTGAGTTTGGCAGCCTCCTCCACGA |
| | GTTCGGCCTTCTGGAAAGCCCCAAGGCCCTGGAGGAGGCCCCCTGGCCCCCGCCGGAAGGGG |
| | CCTTCGTGGGCTTTGTGCTTTCCCGCAAGGAGCCCATGTGGGCCGATCTTCTGGCCCTGGCC |
| | GCCGCCAGGGGGGGCCGGGTCCACCGGGCCCCCGAGCCTTATAAAGCCCTCAGGGACCTGAA |
| | GGAGGCGCGGGGGCTTCTCGCCAAAGACCTGAGCGTTCTGGCCCTGAGGGAAGGCCTTGGCC |
| | TCCCGCCCGGCGACGACCCCATGCTCCTCGCCTACCTCCTGGACCCTTCCAACACCACCCCC |
| | GAGGGGTGGCCCGGGCGCTACGGCGGGGAGTGGACGGAGGAGGCGGGGGAGCGGGCCGCCCT |
| | TTCCGAGAGGCTCTTCGCCAACCTGTGGGGGAGGCTTGAGGGGGAGGAGAGGCTCCTTTGGC |
| | TTTACCGGGAGGTGGAGAGGCCCCTTTCCGCTGTCCTGGCCCACATGGAGGCCACGGGGGTG |
| | CGCCTGGACGTGGCCTATCTCAGGGCCTTGTCCCTGGAGGTGGCCGAGGAGATCGCCCGCCT |
| | CGAGGCCGAGGTCTTCCGCCTGGCCGGCCACCCCTTCAACCTCAACTCCCGGGACCAGCTGG |
| | AAAGGGTCCTCTTTGACGAGCTAGGGCTTCCCGCCATCGGCAAGACGGAGAAGACCGGCAAG |
| | CGCTCCACCAGCGCCGCCGTCCTGGAGGCCCTCCGCGAGGCCCACCCCATCGTGGAGAAGAT |
| | CCTGCAGTACCGGGAGCTCACCAAGCTGAAGAGCACCTACATTGACCCCTTGCCGGACCTCA |
| | TCCACCCCAGGACGGGCCGCCTCCACACCCGCTTCAACCAGACGGCCACGGCCACGGGCAGG |
| | CTAAGTAGCTCCGATCCCAACCTCCAGAACATCCCCGTCCGCACCCCGCTTGGGCAGAGGAT |
| | CCGCCGGGCCTTCATCGCCGAGGAGGGGTGGCTATTGGTGGCCCTGGACTATAGCCAGATAG |
| | AGCTCAGGGTGCTGGCCCACCTCTCCGGCGACGAGAACCTGATCCGGGTCTTCCAGGAGGGG |
| | CGGGACATCCACACGGAGACCGCCAGCTGGATGTTCGGCGTCCCCCGGGAGGCCGTGGACCC |
| | CCTGATGCGCCGGGCGGCCAAGACCATCAACTTCGGGGTCCTCTACGGCATGTCGGCCCACC |
| | GCCTCTCCCAGGAGCTAGCCATCCCTTACGAGGAGGCCCAGGCCTTCATTGAGCGCTACTTT |
| | CAGAGCTTCCCCAAGGTGCGGGCCTGGATTGAGAAGACCCTGGAGGAGGGCAGGAGGCGGGG |
| | GTACGTGGAGACCCTCTTCGGCCGCCGCCGCTACGTGCCAGACCTAGAGGCCCGGGTGAAGA |
| | GCGTGCGGGAGGCGGCCGAGCGCATGGCCTTCAACATGCCCGTCCAGGGCACCGCCGCCGAC |
| | CTCATGAAGCTGGCTATGGTGAAGCTCTTCCCCAGGCTGGAGGAAATGGGGGCCAGGATGCT |
| | CCTTCAGGTCCACGACGAGCTGGTCCTCGAGGCCCCAAAAGAGAGGGCGGAGGCCGTGGCCC |
| | GGCTGGCCAAGGAGGTCATGGAGGGGGTGTATCCCCTGGCCGTGCCCCTGGAGGTGGAGGTG |
| | GGGATAGGGGAGGACTGGCTCTCCGCCAAGGAGTAATGA |

### Example 2

### Use of Protamine Coated Beads for DNA Contaminant Removal

For protamine attachment, NHS-activated Sepharose beads were utilized and coupling was performed in 0.1 M HEPES buffer (pH 7.0), with a protein to bead ratio of 25 mg of the protein to 1 ml bead suspension (0.5 ml packed), pre-washed with 1 mM HCl. Attachment of protamine to beads was completed by leaving the suspension on rotation overnight at 4° C., followed by washing with 0.1 M Tris (pH 8.5), to block any remaining NHS groups on beads. The procedure ensured overall coverage of Sepharose beads with the highly charged protamine molecules.

DNA contaminant removal was accomplished using 15 μl of packed protamine-Sepharose beads per mg Taq polymerase, in 0.05 M sodium acetate, 0.2 M potassium chloride, pH 5.0. After leaving on rotation for 10 minutes, the suspension was centrifuged for 3 minutes at 1000×g, and the supernatant containing the enzyme was tested for DNA contamination, using a Qubit quantitation system.

An optional step was carried out after the protamine-based step to further remove nucleic acid contaminant. In this step, the affinity beads containing Taq polymerase was placed into a dialysis bag (MWCO 1000 kD) and a voltage (45 V) was applied for 10 minutes. The buffer condition was 100 mM MES, pH 5.3.

### Example 3

### DNA Contaminant Removal using Silica Resin

In some instances, silica resin is used for removal of DNA from the growth media employed for enzyme production. Sometimes, treatment of growth media with silica resin can remove greater than 90% of DNA contaminant. For each 100 ml of growth medium, 0.1 g of silica was used for DNA removal, upon shaking the mixture for 2 hours at room temperature, followed by centrifugation.

### Example 4

### Characterization of Polymerase Activity

The assay was fixed at a final volume of 100 μl, and contained reaction components at the following final concentrations: oligonucleotide of SEQ ID NO: 8; 40 μM, dNTPs; 200 μM (each), APS; 1.6 mM, ATP sulfurylase; 0.12 Units, D-Luciferin; 0.3 mM, and Luciferase; 3 μg.

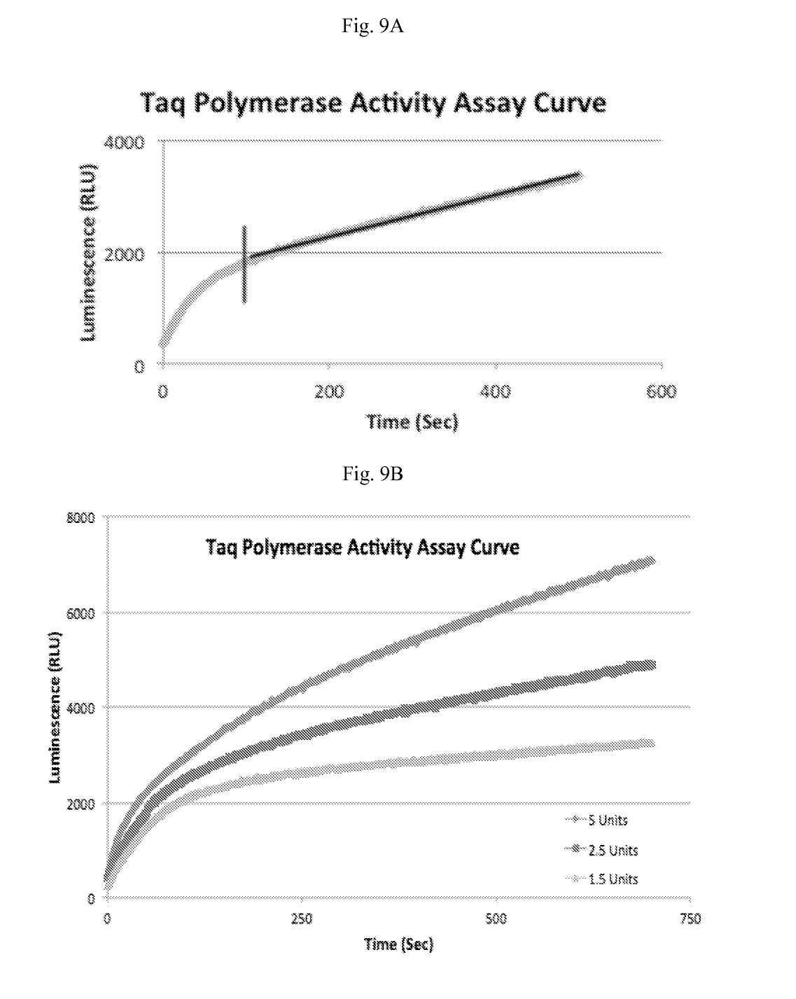

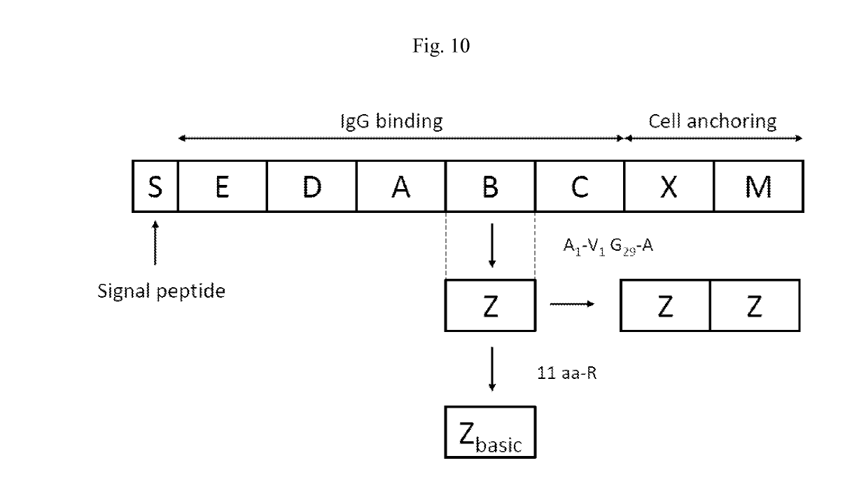

FIG. 9 illustrates a luminescence assay that indicates the Taq polymerase activity. As shown in FIG. 9A and FIG. 9B, a time course is initially indicated with a lag phase, normally less than 2 minutes, followed by a linear increase in luminescence. The rate of luminescence increase (slope) defines the Taq polymerase activity.

### Characterization of Polymerase Activity in the Presence of a Polymerase Inhibitor

Hot Start Taq DNA polymerase was obtained from New England BioLabs (NEB). Taq polymerase from New England BioLabs was tested as a control. An amplification reaction was performed to determine the activity of Bio-HIS-ABS-Taq in the presence of human serum albumin (HSA) at 45° C. and to compare the activity of Bio-HIS-ABS-Taq in the presence of HSA with Hot Start Taq polymerase (NEB). The ratio of Bio-HIS-ABS-Taq to HSA is 1:1. Table 2 illustrates the activity of Bio-HIS-ABS-Taq in the presence of HSA compared to the activity of Hot Start Taq Polymerase from NEB.

TABLE 2

| | % activity | | % activity |
|---|---|---|---|
| Control #1 (Taq, NEB) | 100 | Control #2 (Taq, | 100 |
| | | NEB) | |
| Bio-HIS-ABS-Taq with | 73 | Hot Start (NEB) | 91 |
| HSA mix (1:1) | | | |

### Example 5

### Optimization of the Protamine-Based Method

pH Optimization: pH at 4.4, 4.75, 5.5, 6, and 7.7 were tested. Buffers used for adjusting the pH included acetate pH 4, acetate pH 5, MES pH 6, HEPES pH 7, and Tris pH 8. In each tube, 4 ul of the protamine bead slurry (protamine from Sigma-Aldrich) was used, 10 ul of buffer, 7 ul of 3.5 M KCl, and 140 ul of the buffer exchanged Bio-HIS-ABS-Taq polymerase. The initial concentrations of the polymerase and DNA contaminant were 0.478 mg/mL and 65.3 ng/mL, respectively. Table 3 illustrates the final concentrations of the polymerase and DNA contaminant.

TABLE 3

| | Qubit DNA | % DNA remaining | Qubit protein | % protein re- |
|---|---|---|---|---|
| | remaining | (comp to buffer | remaining | maining (comp |
| pH | (ng/ml) | exchanged) | (mg/ml) | to buffer exch) |
| 4.4 | <5.75 | <8.8 | 0.462 | 97 |
| 4.75 | <5.75 | <8.8 | 0.288 | 60 |
| 5.5 | 7.01 | 10.7 | 0.292 | 61 |
| 6 | 10.35 | 15.8 | 0.308 | 64 |
| 7.7 | 17.135 | 26.2 | 0.360 | 75 |

Table 4 illustrates the final concentrations of the Bio-HIS-ABS-Taq polymerase and DNA contaminant from a second set of experiment. The initial concentrations of the polymerase and DNA contaminant were 0.55 mg/mL and 65.3 ng/mL, respectively.

TABLE 4

| | Qubit DNA | % DNA remaining | Qubit protein | % protein re- |
|---|---|---|---|---|
| | remaining | (comp to buffer | remaining | maining (comp |
| pH | (ng/ml) | exchanged) | (mg/ml) | to buffer exch) |
| 4.03 | <5.6 | <8.6 | 0.459 | 94 |
| 4.22 | <5.4 | <8.2 | 0.486 | 95 |
| 4.40 | <5.2 | <8.0 | 0.505 | 96 |
| 4.80 | 25.5 | 39.1 | 0.497 | 93 |

Table 5 illustrates the final concentrations of the Bio-HIS-ABS-Taq polymerase and DNA contaminant from an experiment that modulates the salt concentration. The initial concentrations of the polymerase and DNA contaminant were 0.487 mg/mL and 82.9 ng/mL, respectively.

TABLE 5

| | Qubit DNA | % DNA remaining | Qubit protein | % protein re- |
|---|---|---|---|---|
| KCl | remaining | (comp to buffer | remaining | maining (comp |
| (M) | (ng/ml) | exchanged) | (mg/ml) | to buffer exch) |
| 0.145 | <5.37 | <6.5 | 0.513 | 105 |
| 0.194 | <5.44 | <6.6 | 0.495 | 101 |
| 0.242 | <5.53 | <6.7 | 0.487 | 99.9 |
| 0.291 | <5.61 | <6.8 | 0.339 | 70 |
| 0.340 | <5.70 | <6.9 | 0.271 | 55 |

Table 6 illustrates the final DNA contaminant concentrations of the Bio-HIS-ABS-Taq polymerase and additional polymerases after the protamine-based method.

TABLE 6

| | ng/mL | Dilution factor | Total (ng/mL) |
|---|---|---|---|
| NEB Taq | 5.2 | 10 | 52 |
| Bio-HIS-ABS-Taq | 2.57 | 10 | 25.7 |
| Biotium Cheetah | 2.45 | 10 | 24.5 |
| Qiagen Hotstar | 7.4 | 10 | 74 |
| AmpliGold 360 | 1.25 | 13.33 | 16.67 |

The examples and embodiments described herein are for illustrative purposes only and various modifications or changes suggested to persons skilled in the art are to be included within the spirit and purview of this application and scope of the appended claims.

What is claimed is:

1. A composition comprising a polymerase comprising at least one albumin binding moiety.

2. The composition of claim 1, wherein the polymerase is a DNA polymerase or an RNA polymerase.

3. The composition of claim 2, wherein the polymerase comprises Taq polymerase, Vent polymerase, Klenow Fragment (3′-5′ exo-), DNA Polymerase I (large Klenow fragment), E. coli DNA polymerase I, phi29 DNA polymerase, Phusion DNA polymerase, or T4 DNA polymerase.

4. The composition of claim 3, wherein the polymerase is Taq polymerase.

5. The composition of claim 4, wherein the Taq polymerase is native or modified Taq polymerase.

6. The composition of claim 4 or 5, wherein the Taq polymerase further comprises a HIS moiety, a biotin-tag moiety, Z domain moiety, or combinations thereof.

7. The composition of claim 6, wherein the at least one albumin binding moiety is directly connected to the Taq polymerase or is connected to the Taq polymerase though a spacer.

8. The composition of claim 6 or 7, wherein a genetic sequence of the at least one albumin binding moiety and the Taq polymerase comprises the at least one albumin binding moiety sequence residing on the 3′ end of the Taq polymerase sequence, residing on the 5′ end of the Taq polymerase sequence, or residing on both the 3′ end and 5′ end of the Taq polymerase sequence.

9. The composition of claim 1, wherein the composition further comprises an albumin.

10. The composition of claim 9, wherein the albumin inhibits the activity of the polymerase by binding to the polymerase at a temperature of from about 0° C. to about 60° C., from about 20° C. to about 55° C., or from about 25° C. to about 50° C.

11. The composition of claim 9 or 10, wherein the albumin is inactivated at a temperature of at least 61° C. or higher.

12. The composition of any one of the claims 9-11, wherein the polymerase regains its enzymatic activity at a temperature of at least 61° C. or higher.

13. The composition of any one of the claims 9-12, wherein the albumin inhibits the activity of the polymerase by about 10% to about 100% relative to a control.

14. The composition of claim 13, wherein the control is the activity of an equivalent polymerase in the absence of a polymerase inhibitor.

15. The composition of any one of the claims 9-14, wherein the albumin is mammalian albumin or a mammalian albumin analogue.

16. The composition of any one of the claims 9-15, wherein the albumin is human serum albumin.

17. The composition of any one of the claims 9-15, wherein the albumin is bovine serum albumin.

18. The composition of any one of the claims 1-17, wherein the albumin binding moiety able to bind serum albumin is at least a part of Streptococcal protein G.

19. The composition of claim 18, wherein the at least a part of Streptococcal protein G is the entire Streptococcal protein G.

20. The composition of claim 18, wherein the at least a part of Streptococcal protein G comprises ABP (121aa), BB (214aa), ABD (46aa), ADB1 binding site, ADB2 binding site, or ADB3 binding site.

21. The composition of claim 20, wherein the ABD to albumin affinity is 1.5 nanomolar or less.

22. The composition of claim 20, wherein the ABD to human serum albumin affinity is 1.5 nanomolar or less.

23. The composition of any one of the claims 1-22, wherein the polymerase has the sequence as illustrated in SEQ ID NO: 1.

24. The composition of any one of the claims 1-23, wherein the polymerase is expressed in an eukaryotic cell.

25. The composition of claim 24, wherein the eukaryotic cell is a yeast cell.

26. The composition of claim 25, wherein the yeast is Pichia pastoris.

27. The composition of any one of the claims 1-23, wherein the polymerase is expressed in E. coli.

28. The composition of any one of the claims 1-27, wherein the polymerase has less than about 30 ng/mL of nucleic acid contaminant.

29. A reaction mixture comprising:
a) a polymerase comprising an albumin binding moiety; and
b) an albumin.

30. The reaction mixture of claim 29, wherein the albumin inhibits the activity of the polymerase by binding to the polymerase at a temperature of from about 0° C. to about 60° C., from about 20° C. to about 55° C., or from about 25° C. to about 50° C.

31. The reaction mixture of claim 29 or 30, wherein the albumin is released from the polymerase at a temperature of at least 61° C. or higher.

32. The reaction mixture of any one of the claims 29-31, wherein the polymerase regains its enzymatic activity at a temperature of at least 61° C. or higher.

33. The reaction mixture of any one of the claims 29-32, wherein the albumin inhibits the activity of the polymerase by about 10% to about 100% relative to a control.

34. The reaction mixture of claim 33, wherein the control is the activity of an equivalent polymerase in the absence of a polymerase inhibitor.

35. The reaction mixture of any one of the claims 29-34, wherein the polymerase is a DNA polymerase or an RNA polymerase.

36. The reaction mixture of any one of the claims 29-35, wherein the polymerase comprises Taq polymerase, Vent polymerase, Klenow Fragment (3′-5′ exo-), DNA Polymerase I (large Klenow fragment), E. coli DNA polymerase I, phi29 DNA polymerase, Phusion DNA polymerase, or T4 DNA polymerase.

37. The reaction mixture of any one of the claims 29-36, wherein the polymerase is Taq polymerase.

38. The reaction mixture of claim 37, wherein the Taq polymerase is native or modified Taq polymerase.

39. The reaction mixture of claim 29, wherein the albumin binding moiety is directly connected to the Taq polymerase or is connected to the Taq polymerase though a spacer.

40. The reaction mixture of claim 39, wherein a genetic sequence of the albumin binding moiety and the Taq polymerase comprises the albumin binding moiety sequence residing on the 3′ end of the Taq polymerase sequence, residing on the 5′ end of the Taq polymerase sequence, or residing on both the 3′ end and 5′ end of the Taq polymerase sequence.

41. The reaction mixture of any one of the claims 29-40, wherein the albumin is mammalian albumin or a mammalian albumin analogue.

42. The reaction mixture of any one of the claims 29-41, wherein the albumin is human serum albumin.

43. The reaction mixture of any one of the claims 29-41, wherein the albumin is bovine serum albumin.

44. The reaction mixture of any one of the claims 29-43, wherein the albumin binding moiety able to bind serum albumin is at least a part of Streptococcal protein G.

45. The reaction mixture of claim 44, wherein the at least a part of Streptococcal protein G is the entire Streptococcal protein G.

46. The reaction mixture of claim 44, wherein the at least a part of Streptococcal protein G comprises ABP (121aa), BB (214aa), ABD (46aa), ADB1 binding site, ADB2 binding site, or ADB3 binding site.

47. The reaction mixture of claim 46, wherein the ABD to albumin affinity is 1.5 nanomolar or less.

48. The reaction mixture of claim 46, wherein the ABD to human serum albumin affinity is 5 nanomolar or less.

49. The reaction mixture of any one of the claims 29-48, wherein the polymerase further comprises a HIS moiety, a biotin-tag moiety, Z domain moiety, or a combination thereof.

50. The reaction mixture of any one of the claims 29-49, wherein the polymerase has the sequence as illustrated in SEQ ID NO: 1.

51. The reaction mixture of any one of the claims 29-50, wherein the reaction mixture is an amplification reaction mixture.

52. The reaction mixture of claim 51, wherein the amplification is a polymerase chain reaction (PCR).

53. The reaction mixture of claim 51 or 52, wherein the amplification comprises whole genome amplification, helicase dependent amplification, nicking enzyme amplification reaction, reverse transcription PCR (RT-PCR), ligation mediated PCR, methylation specific PCR, digital PCR, hot start PCR, multiplex ligation-dependent probe amplification (MLPA), multiplex-PCR, nested PCR, overlap-extension PCR, or quantitative PCR (qPCR).

54. The reaction mixture of claim 51 or 52, wherein the amplification is a next-generation sequencing method.

55. A polymerase construct comprising at least one moiety that is capable of binding albumin.

56. The polymerase construct of claim 55, wherein the polymerase is a DNA polymerase or an RNA polymerase.

57. The polymerase construct of claim 55 or 56, wherein the polymerase comprises Taq polymerase, Vent polymerase, Klenow Fragment (3′-5′ exo-), DNA Polymerase I (large Klenow fragment), E. coli DNA polymerase I, phi29 DNA polymerase, Phusion DNA polymerase, or T4 DNA polymerase.

58. The polymerase construct of claim 57, wherein the polymerase is Taq polymerase.

59. The polymerase construct of claim 58, wherein the Taq polymerase is native or modified Taq polymerase.

60. The polymerase construct of any one of the claims 55-59, wherein the at least one moiety is directly connected to the Taq polymerase or is connected to the Taq polymerase though a spacer.

61. The polymerase construct of any one of the claims 55-60, wherein a genetic sequence of the at least one moiety and the Taq polymerase comprises the at least one moiety sequence residing on the 3′ end of the Taq polymerase sequence, residing on the 5′ end of the Taq polymerase sequence, or residing on both the 3′ end and 5′ end of the Taq polymerase sequence.

62. The polymerase construct of any one of the claims 55-61, wherein the albumin is mammalian albumin or a mammalian albumin analogue.

63. The polymerase construct of any one of the claims 55-62, wherein the albumin is human serum albumin or bovine serum albumin.

64. The polymerase construct of any one of the claims 55-63, wherein the at least one moiety bind to serum albumin.

65. The polymerase construct of any one of the claims 55-64, wherein the at least one moiety able to bind serum albumin is at least a part of Streptococcal protein G.

66. The polymerase construct of claim 65, wherein the at least a part of Streptococcal protein G is the entire Streptococcal protein G.

67. The polymerase construct of claim 65, wherein the at least a part of Streptococcal protein G comprises ABP (121aa), BB (214aa), ABD (46aa), ADB1 binding site, ADB2 binding site, or ADB3 binding site.

68. The polymerase construct of claim 67, wherein the ABD to albumin affinity is 1.5 nanomolar or less.

69. The polymerase construct of claim 67, wherein the ABD to human serum albumin affinity is 1.5 nanomolar or less.

70. The polymerase construct of any one of the claims 55-69, wherein the polymerase construct further comprises a HIS moiety, a biotin-tag moiety, Z domain moiety, or a combination thereof.

71. The polymerase construct of any one of the claims 55-70, wherein the polymerase construct is the construct as illustrated in SEQ ID NO: 1.

72. A method for amplifying a target DNA comprising:
a) incubating the target DNA with a polymerase having the polymerase construct of claims 55-71, an albumin, a set of primers, and nucleoside phosphates selected from the group consisting of adenine, thymine, guanine, cytosine, and uridine; so as to form a reaction mixture; and
b) subjecting the reaction mixture to an amplification method, whereby the set of primers is extended by the polymerase to amplify the target DNA sequence.

73. The method of claim 72, wherein the albumin inhibits the activity of the polymerase by binding to the polymerase at a temperature of from about 0° C. to about 60° C., from about 20° C. to about 55° C., or from about 25° C. to about 50° C.

74. The method of claim 72 or 73, wherein the albumin is inactivated at a temperature of at least 61° C. or higher.

75. The method of any one of the claims 72-74, wherein the polymerase regains its enzymatic activity at a temperature of at least 61° C. or higher.

76. The method of claim 72, wherein the polymerase is expressed in an eukaryotic cell.

77. The method of claim 76, wherein the polymerase is expressed in Pichia pastoris.

78. The method of claim 72, wherein the amplification method is a polymerase chain reaction (PCR).

79. An albumin affinity separation method for enzyme purification comprising:
a) forming a protein construct comprising a target polymerase bound to an albumin binding moiety;
b) contacting the protein construct with albumin to form an albumin molecular complex;
c) separating the protein construct; and
d) retrieving the protein construct from the albumin molecular complex, wherein the protein construct retains activity of the target polymerase.

80. The method of claim 79, wherein the protein construct further comprises a HIS binding moiety.

81. The method of claim 79 or 80, wherein the method further comprises purifying the protein construct using a HIS affinity separation method.

82. The method of claim 81, wherein the HIS affinity separation method comprises:
a) contacting the protein construct with HIS binding moiety to form a HIS molecular complex;
b) separating the protein construct from species not bound to HIS binding moiety; and
c) retrieving the protein construct from the HIS molecular complex, wherein the protein construct retains the activity of the target polymerase.

83. The method of claim 82, wherein the HIS affinity separation method precedes the albumin affinity separation method, or the albumin affinity separation method precedes the His affinity separation method.

84. The method of claim 79, wherein the protein construct further comprises a biotin-tag moiety, a Z domain moiety, or a combination thereof.

85. The method of claim 79, wherein the retrieving comprises altering the pH of at least the environment immediately surrounding the albumin molecular complex.

86. The method of claim 85, wherein the altering the pH is elevating the pH value of at least the environment immediately surrounding the albumin molecular complex, or reducing the pH value of at least the environment immediately surrounding the albumin molecular complex.

87. The method of claim 79, wherein the retrieving comprises altering the salt concentration of at least the environment immediately surrounding the albumin molecular complex, altering the conductivity of at least the environment immediately surrounding the albumin molecular complex, altering the temperature of at least the environment immediately surrounding the albumin molecular complex, or a combination thereof.

88. The method of claim 79, wherein the separating comprises washing with an aqueous solution.

89. The method of claim 79, wherein the albumin is mammalian albumin or a mammalian albumin analogue.

90. The method of claim 79 or 89, wherein the albumin is human serum albumin or bovine serum albumin.

91. The method of any one of the claims 79-90 wherein the albumin is bound to a solid support or bound to particles.

92. The method of claim 91, wherein the particles are assembled into a column.

93. The method of claim 91 or 92, wherein the particles are magnetic particles.

94. The method of claim 91, wherein the bond is covalently bound.

95. The method of claim 94, wherein the covalently bound is direct or indirect through a molecular spacer.

96. The method of claim 79, wherein the polymerase is a DNA polymerase or an RNA polymerase.

97. The method of claim 79 or 96, wherein the polymerase is Taq polymerase.

98. The method of claim 97, wherein the Taq polymerase is native or modified Taq polymerase.

99. The method of any one of the claims 79-98, wherein the albumin binding moiety is directly connected to the Taq polymerase or the albumin binding moiety is connected to the Taq polymerase though a spacer.

100. The method of any one of the claims 79-99, wherein a genetic sequence of the albumin binding moiety and the Taq polymerase comprises the albumin binding moiety sequence residing on the 3′ end of the Taq polymerase sequence, residing on the 5′ end of the Taq polymerase sequence, or residing on both the 3′ end and 5′ end of the Taq polymerase sequence.

101. The method of any one of the claims 79-100, wherein the Taq polymerase consists of a sequence selected from SEQ ID NO: 1.

102. The method of any one of the claims 79-101, wherein the albumin binding moiety able to bind serum albumin is at least a part of Streptococcal protein G.

103. The method of claim 102, wherein the at least a part of Streptococcal protein G is the entire Streptococcal protein G.

104. The method of claim 102, wherein the at least a part of Streptococcal protein G comprises ABP (121aa), BB (214aa), ABD (46aa), ADB1 binding site, ADB2 binding site, or ADB3 binding site.

105. The method of claim 104, wherein the ABD to albumin affinity is 1.5 nanomolar or less.

106. A method of removing a nucleic acid contaminant from a biological sample, comprising:
a) contacting a biological sample with protamine-coated beads; and
b) harvesting the biological sample from protamine-coated beads through a separation method to remove the nucleic acid contaminant from the biological sample.

107. The method of claim 106, wherein the biological sample is a protein sample.

108. The method of claim 106 or 107, wherein the biological sample is a polymerase sample.

109. The method of claim 108, wherein the polymerase sample is a DNA polymerase sample or an RNA polymerase sample.

110. The method of claim 108 or 109, wherein the polymerase sample is a Taq polymerase sample.

111. The method of claim 110, wherein the Taq polymerase is native or modified Taq polymerase.

112. The method of claim 111, wherein the Taq polymerase consists of a sequence selected from SEQ ID NO: 1.

113. The method of claim 106, wherein the biological sample is a cell lysis sample.

114. The method of claim 106, wherein the biological sample is a culture media sample.

115. The method of claim 106, wherein the protamine-coated beads are beads covalently bound to protamine.

116. The method of claim 106 or 115, wherein the beads are Sepharose beads or magnetic beads.

117. The method of claim 106, wherein the contacting comprises incubating the biological sample with protamine-coated beads for from about 2 min to about 24 hours.

118. The method of claim 106 or 117, wherein the contacting further comprises incubating the biological sample with protamine-coated beads at a buffer pH of from about 4 to about 9.

119. The method of any one of the claim 106, 117, or 118, wherein the contacting further comprises incubating about 1 μL to about 100 μL of protamine-coated beads with about 1 mg of biological sample.

120. The method of claim 106, wherein the separation method is a centrifugation method or column chromatography method.

121. The method of claim 106, wherein the nucleic acid contaminant is DNA contaminant.

122. The method of claim 106, wherein the method further comprises removing the nucleic acid contaminant through an electrophoretic method.

123. The method of claim 122, wherein the electrophoretic method is performed after harvesting the biological sample from the protamine-coated beads.

124. The method of claim 106 or 122, wherein the method further comprises removing the nucleic acid contaminant through a silica-based method.

125. The method of claim 124, wherein the silica-based method comprises contacting a growth media with silica and harvesting the silica-treated growth media with a separation method.

126. The method of any one of the claims 106-125, wherein protamine is obtained from salmon.

127. An assay kit for determining the activity of a polymerase comprising an oligonucleotide selected from SEQ ID NOs: 8-10.

128. The assay kit of claim 127, wherein the polymerase is a DNA polymerase or an RNA polymerase.

129. The assay kit of claim 127 or 128, wherein the polymerase comprises Taq polymerase, Vent polymerase, Klenow Fragment (3′-5′ exo-), DNA Polymerase I (large Klenow fragment), E. coli DNA polymerase I, phi29 DNA polymerase, Phusion DNA polymerase, or T4 DNA polymerase.

130. The assay kit of claim 129, wherein the polymerase is a Taq polymerase.

131. The assay kit of claim 130, wherein the Taq polymerase is native or modified Taq polymerase.

132. The assay kit of any one of the claims 127-131, wherein the assay kit further comprises a primer.

133. The assay kit of claim 127, wherein the activity of the polymerase is determined from an amplification reaction.

134. The assay kit of claim 133, wherein a pyrophosphate is released during the amplification reaction.

135. The assay kit of claim 134, wherein the rate of pyrophosphate release during the amplification reaction is used to determine the activity of the polymerase.


SEQUENCE LISTING (13 sequences)

SEQ ID NO: 1 - protein, 1003 aa, Artificial Sequence
  Synthetic polypeptide Bio-HIS-ABS-Taq polymerase
MASSLRQILDSQKIEWRSNAGGASHHHHHHGGASLAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPGTFAHYMDPNLEALFQGPNSLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGWPGRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 2 - DNA, 3015 bp, Artificial Sequence
  Synthetic polynucleotide
atggctagtagcctgcgccagatcctggacagccagaaaatcgaatggcgcagcaacgctggtggtgctagtcaccaccaccaccaccacggtggtgctagcttagctgaagctaaagtcttagctaacagagaacttgacaaatatggagtaagtgactatcacaagaacctaatcaacaatgccaaaactgttgaaggtgtaaaagaccttcaagcacaagttgttgaatcagcgaagaaagcgcgtatttcagaagcaacagatggcttatctgatttcttgaaatcacaaacacctgctgaagatactgttaaatcaattgaattagctgaagctaaagtcttagctaacagagaacttgacaaatatggagtaagtgactattacaagaacctaatcaacaatgccaaaactgttgaaggtgtaaaagcactgatagatgaaattttagctgcattacctggtaccttcgctcactacatggatccgaatttggaagctctgttccagggtccgaattcgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgaggggtggcccgggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtaatga

SEQ ID NO: 3 - protein, 24 aa, Artificial Sequence
  Synthetic peptide
MASSLRQILDSQKIEWRSNAGGAS

SEQ ID NO: 4 - DNA, 72 bp, Artificial Sequence
  Synthetic polynucleotide
atggctagtagcctgcgccagatcctggacagccagaaaatcgaatggcgcagcaacgctggtggtgctagt

SEQ ID NO: 5 - protein, 15 aa, Artificial Sequence
  Synthetic peptide
GLNDIFEAQKIEWHE

SEQ ID NO: 6 - DNA, 27 bp, Artificial Sequence
  Synthetic polynucleotide
tttttgcatggtaattcgtcagactgg

SEQ ID NO: 7 - DNA, 43 bp, Artificial Sequence
  Synthetic polynucleotide
gccgtcgcgcttttacaacggaacgttgtaaaagcgcgacggc

SEQ ID NO: 8 - DNA, 70 bp, Artificial Sequence
  Synthetic polynucleotide
tttttgcatggtaattcgtcagactgggccgtcgcgcttttacaacggaacgttgtaaaagcgcgacggc

SEQ ID NO: 9 - DNA, 60 bp, Artificial Sequence
  Synthetic polynucleotide
tttttgctggaattcgtcagactggccgtcgttttacaacggaacgttgtaaaacgacgg

SEQ ID NO: 10 - DNA, 60 bp, Artificial Sequence
  Synthetic polynucleotide
tttttccccctttttgggggaaaaaccgtcgttttacaacggaacgttgtaaaacgacgg

SEQ ID NO: 11 - protein, 57 aa, Artificial Sequence
  Synthetic polypeptide
VDNKFNKEQQNAFYEILHLPNLNEEQRNAFIQSLKDDPSQSANLLAEAKKLNDAQPK

SEQ ID NO: 12 - protein, 57 aa, Artificial Sequence
  Synthetic polypeptide
VDNKFNKEEEEAEEEIEELPNLNEEQEEAFIESLEDDPSQSANLLAEAKKLNDAQPK

SEQ ID NO: 13 - protein, 57 aa, Artificial Sequence
  Synthetic polypeptide
VDNKFNKERRRARREIRHLPNLNEEQRRAFIRSLRDDPSQSANLLAEAKKLNDAQPK